(19) United States
(12) Patent Application Publication
(10) Pub. No.: US 2024/0169671 A1
(43) Pub. Date: May 23, 2024

(54) MEDICAL INFORMATION PROCESSING APPARATUS, MEDICAL INFORMATION PROCESSING METHOD, RECORDING MEDIUM, AND INFORMATION PROCESSING APPARATUS

(71) Applicant: CANON MEDICAL SYSTEMS CORPORATION, Tochigi (JP)

(72) Inventor: Gakuto AOYAMA, Otawara (JP)

(73) Assignee: CANON MEDICAL SYSTEMS CORPORATION, Tochigi (JP)

(21) Appl. No.: 18/510,781

(22) Filed: Nov. 16, 2023

(30) Foreign Application Priority Data
Nov. 17, 2022 (JP) 2022-184095

(51) Int. Cl. G06T 17/20; G06T 7/60; G06T 7/70; G06V 20/50; G16H 40/63; G16H 50/50

(52) U.S. Cl. CPC G06T 17/20; G06T 7/60; G06T 7/70; G06V 20/50; G16H 40/63; G16H 50/50; G06T 2207/10024; G06T 2207/10028; G06T 2207/30004; G06T 2210/41; G06T 2210/56; G06V 2201/031

(57) ABSTRACT

A medical information processing apparatus according to an embodiment includes processing circuitry that is configured to acquire medical image data that includes a target organ, acquire grid point cloud data that is associated with the medical image data and that is related to the target organ, display the medical image data, and that identify an attention grid included in the grid point cloud data on the basis of a display condition of the medical image data.

### CROSS-REFERENCE TO RELATED APPLICATIONS

This application is based upon and claims the benefit of priority from Japanese Patent Application No. 2022-184095, filed on Nov. 17, 2022; the entire contents of which are incorporated herein by reference.

### FIELD

Embodiments described herein relate generally to a medical information processing apparatus, a medical information processing method, a recording medium, and an information processing apparatus.

### BACKGROUND

Conventionally, a physical simulation performed by using grid point cloud data related to a target object, such as an organ, is used for various purposes. For example, before treatment, by performing the physical simulation using the grid point cloud data related to the target organ that is to be subjected to treatment, it is possible to estimate a state of the target organ at the time of post-treatment.

### BRIEF DESCRIPTION OF THE DRAWINGS

FIG. 1 is a block diagram illustrating one example of a configuration of a medical information processing system according to an embodiment;

FIG. 2 is a flowchart illustrating one example of a process performed by processing circuitry included in a medical information processing apparatus according to the embodiment;

FIG. 3A is a diagram illustrating one example of grid point cloud data according to the embodiment;

FIG. 3B is a diagram illustrating one example of grid point cloud data according to the embodiment;

FIG. 4A is a diagram illustrating one example of grid point cloud data according to the embodiment;

FIG. 4B is a diagram illustrating a structure of a mitral valve according to the embodiment;

FIG. 5A is a display example according to the embodiment;

FIG. 5B is a diagram for explaining a mesh editing function according to the embodiment;

FIG. 5C is a diagram for explaining the mesh editing function according to the embodiment;

FIG. 5D is a diagram for explaining the mesh editing function according to the embodiment;

FIG. 6A is a display example obtained when a three-dimensional mesh is superimposed on a two-dimensional image according to the embodiment;

FIG. 6B is a display example obtained when a three-dimensional mesh is superimposed on a two-dimensional image according to the embodiment;

FIG. 6C is a display example obtained when a three-dimensional mesh is superimposed on a two-dimensional image according to the embodiment;

FIG. 7A is a diagram illustrating one example of a setting screen of a display condition according to the embodiment;

FIG. 7B is a diagram illustrating one example of a setting screen of a display condition according to the embodiment;

FIG. 8A is a display example according to the embodiment;

FIG. 8B is one example of an icon according to the embodiment;

FIG. 8C is one example of an icon according to the embodiment;

FIG. 9A is a diagram for explaining a process of identifying an attention grid according to the embodiment;

FIG. 9B is a diagram for explaining the process of identifying an attention grid according to the embodiment;

FIG. 9C is a diagram for explaining the process of identifying an attention grid according to the embodiment;

FIG. 9D is a diagram for explaining the process of identifying an attention grid according to the embodiment;

FIG. 10 is a display example according to the embodiment;

FIG. 11A is a display example of a result obtained from a physical simulation according to the embodiment;

FIG. 11B is a display example of a result obtained from the physical simulation according to the embodiment;

FIG. 11C is a display example of a result obtained from the physical simulation according to the embodiment;

FIG. 12 is a diagram for explaining the process of identifying an attention grid according to the embodiment;

FIG. 13 is a diagram for explaining the process of identifying an attention grid according to the embodiment;

FIG. 14A is a diagram for explaining the process of identifying an attention grid according to the embodiment;

FIG. 14B is a diagram for explaining the process of identifying an attention grid according to the embodiment; and

FIG. 15 is a block diagram illustrating one example of a configuration of an information processing system according to the embodiment.

### DETAILED DESCRIPTION

A medical information processing apparatus according to embodiments comprises processing circuitry configured to acquire medical image data that includes a target organ acquire grid point cloud data that is associated with the medical image data and that is related to the target organ display the medical image data; and identify an attention grid included in the grid point cloud data on the basis of a display condition of the medical image data.

Embodiments of a medical information processing apparatus, a medical information processing method, a recording medium, and an information processing apparatus will be described below with reference to the accompanying drawings.

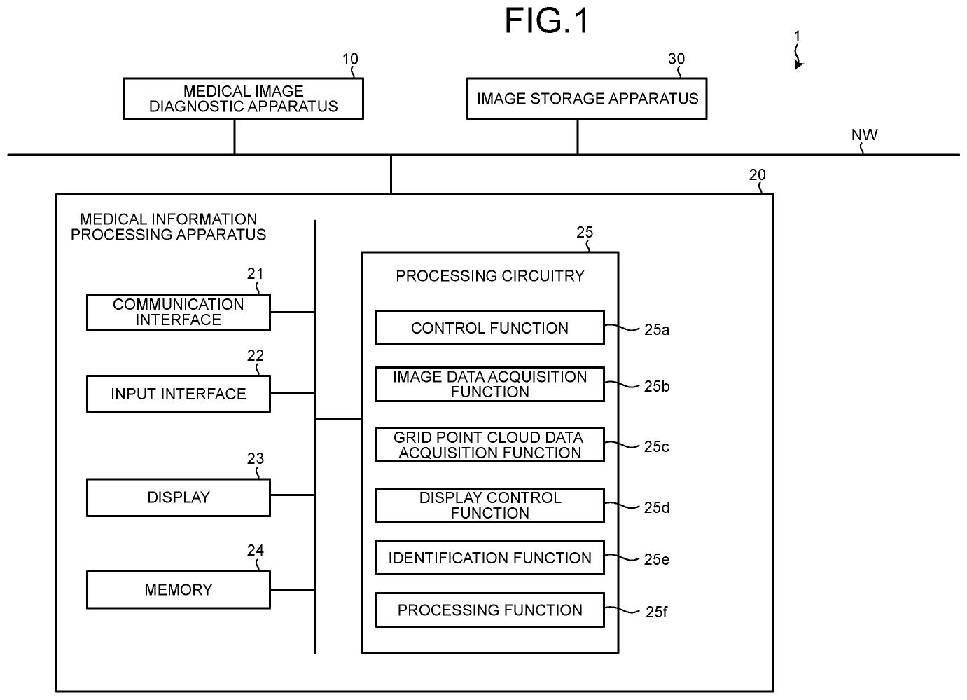

In the present embodiment, a medical information processing system 1 that includes a medical information processing apparatus 20 will be described as an example. For example, as illustrated in FIG. 1, the medical information processing system 1 includes a medical image diagnostic apparatus 10, the medical information processing apparatus 20, and an image storage apparatus 30. FIG. 1 is a block diagram illustrating one example of a configuration of the medical information processing system 1 according to the embodiment. The medical image diagnostic apparatus 10, the medical information processing apparatus 20, and the image storage apparatus 30 are connected with each other via a network NW.

Any location may be used to install each of the apparatuses included in the medical information processing system 1 as long as the apparatuses are able to be connected each other via the network NW. For example, the image storage apparatus 30 may also be installed in a hospital that is different from a hospital in which the medical image diagnostic apparatus 10 and the medical information processing apparatus 20 are installed, or the image storage apparatus 30 may also be installed in another facility. In other words, the network NW may be configured by a local area network that is used in a closed network in a facility, or may also be a network connected via the Internet.

The medical image diagnostic apparatus 10 is a device that captures an image of a subject and that collects medical image data. In addition, various kinds of data handled in the present application are, typically, digital data. The medical image diagnostic apparatus 10 is, for example, a medical modality, such as an X-ray diagnostic apparatus, an X-ray computed tomography (CT) device, a magnetic resonance imaging (MRI) device, an ultrasound diagnostic apparatus, a single photon emission computed tomography (SPECT) device, and a positron emission computed tomography (PET) device. Furthermore, in FIG. 1, the medical image diagnostic apparatus 10 is illustrated as a single unit, but the medical information processing system 1 may also include the plurality of medical image diagnostic apparatuses 10. Moreover, the medical information processing system 1 may also include a plurality of types of the medical image diagnostic apparatuses 10. For example, the medical information processing system 1 may also include an X-ray CT apparatus and an MRI apparatus as the medical image diagnostic apparatus 10.

The image storage apparatus 30 is an image database that stores the medical image data collected by the medical image diagnostic apparatus 10. For example, the image storage apparatus 30 includes an arbitrary storage device that is provided inside the device or outside the device, and manages the medical image data that has been acquired from the medical image diagnostic apparatus 10 via the network NW in the form of a database. For example, the image storage apparatus 30 is a server used for a picture archiving and communication system (PACS). The image storage apparatus 30 may also be implemented by a server group (cloud) that is connected to the medical information processing system 1 via the network NW.

The medical information processing apparatus 20 is an apparatus that acquires the medical image data acquired by the medical image diagnostic apparatus 10, and that performs various kinds of processes. For example, as illustrated in FIG. 1, the medical information processing apparatus 20 includes a communication interface 21, an input interface 22, a display 23, a memory 24, and processing circuitry 25.

The communication interface 21 controls transmission and communication of various kinds of data that are sent and received between the medical information processing apparatus 20 and the other device that is connected by the network NW. Specifically, the communication interface 21 is connected to the processing circuitry 25, and transmits data received from the other device to the processing circuitry 25 or transmits data received from the processing circuitry 25 to the other device. For example, the communication interface 21 is implemented by a network card, a network adapter, a network interface controller (NIC), or the like.

The input interface 22 receives various kinds of input operations from a user, converts the received input operation to an electrical signal, and outputs the converted signal to the processing circuitry 25. For example, the input interface 22 is implemented by a mouse, a keyboard, a trackball, a switch, a button, a joystick, a touch pad with which an input operation is performed by touching an operation surface, a touch screen in which a display screen and a touch pad are integrated, a non-contact input circuit using an optical sensor, a sound input circuit, or the like. In addition, the input interface 22 may be configured by a tablet terminal or the like that is able to perform wireless communication with the main body of the medical information processing apparatus 20. In addition, the input interface 22 may be a circuit that receives an input operation from a user by using a motion capture technology. As one example, by processing signals acquired via a tracker or by processing images collected about a user, the input interface 22 is able to receive a body motion of a user, a line of sight of a user, or the like as an input operation. In addition, the input interface 22 is not limited to the one that includes physical operation parts, such as a mouse and a keyboard. Examples of the input interface 22 also include an electrical signal processing circuit that receives an electrical signal corresponding to an input operation from an external input device that is provided separately from the medical information processing apparatus 20 and outputs this electrical signal to the processing circuitry 25.

The display 23 is, for example, a liquid crystal display or a cathode ray tube (CRT) display. The display 23 may be configured by a desktop type, or may be configured by a tablet terminal or the like that is able to perform wireless communication with the main body of the medical information processing apparatus 20. Control of a display in the display 23 will be described later.

The memory 24 is implemented by, for example, semiconductor memory device, such as a random access memory (RAM) or a flash memory, a hard disk, an optical disk, or the like. For example, the memory 24 stores therein medical image data. Furthermore, the memory 24 also stores therein programs for the circuit included in the medical information processing apparatus 20 to implement functions of the circuit.

The processing circuitry 25 controls the overall operation of the medical information processing apparatus 20 by performing a control function 25a, an image data acquisition function 25b, a grid point cloud data acquisition function 25c, a display control function 25d, an identification function 25e, and a processing function 25f. The image data acquisition function 25b is one example of an image data acquisition unit. The grid point cloud data acquisition function 25c is one example of a grid point cloud data acquisition unit. The display control function 25d is one example of a display control unit. The identification function 25e is one example of an identification unit. The processing function 25f is one example of a processing unit.

For example, the processing circuitry 25 reads the program corresponding to the control function 25a from the memory 24 and executes the read program, thereby controlling various kinds of functions, such as the image data acquisition function 25b, the grid point cloud data acquisition function 25c, the display control function 25d, the identification function 25e, and the processing function 25f, on the basis of various kinds of input operations received from the user via the input interface 22.

In addition, the processing circuitry 25 reads the program corresponding to the image data acquisition function 25b from the memory 24 and executes the read program, thereby acquiring the medical image data including the target organ. Furthermore, the processing circuitry 25 reads the program corresponding to the grid point cloud data acquisition function 25c from the memory 24 and executes the read program, thereby acquiring the grid point cloud data related to the target organ that is associated with the medical image data. In addition, the processing circuitry 25 reads the program corresponding to the display control function 25d from the memory 24 and executes the read program, thereby causing the medical image data to be displayed. In addition, the processing circuitry 25 reads the program corresponding to the identification function 25e from the memory 24 and executes the read program, thereby identifying an attention grid included in the grid point cloud data on the basis of the display condition of the medical image data. Moreover, the processing circuitry 25 reads the program corresponding to the processing function 25f from the memory 24 and executes the read program, thereby performing the physical simulation by using the identified attention grid as a calculation condition. The processes of the image data acquisition function 25b, the grid point cloud data acquisition function 25c, the display control function 25d, the identification function 25e, and the processing function 25f will be described in detail later.

In the medical information processing apparatus 20 illustrated in FIG. 1, each of the processing functions are stored in the memory 24 in the form of a computer-executable program. The processing circuitry 25 is a processor that implements each of the functions corresponding to the programs by reading the program from the memory 24 and executing the read program. In other words, the processing circuitry 25 that has read one of the programs has a function that corresponds to the read program.

In the above, in FIG. 1, the case has been described as an example in which, in the processing circuitry 25 that is a single unit, the control function 25a, the image data acquisition function 25b, the grid point cloud data acquisition function 25c, the display control function 25d, the identification function 25e, and the processing function 25f are implemented, but it may be possible to configure the processing circuitry 25 by a combination of a plurality of independent processors, and cause each of the processors to execute the programs and implement the functions. Furthermore, each of the processing functions included in the processing circuitry 25 may be implemented by being distributed to a plurality of processing circuits or integrated into a single piece of processing circuit as appropriate.

Furthermore, the processing circuitry 25 may implement the functions by using a processor of an external device that is connected via the network NW. For example, the processing circuitry 25 implements each of the functions illustrated in FIG. 1 by reading a program corresponding to each of the functions from the memory 24, and using, as a calculation resource, a server group (cloud) that is connected to the medical information processing apparatus 20 via the network NW.

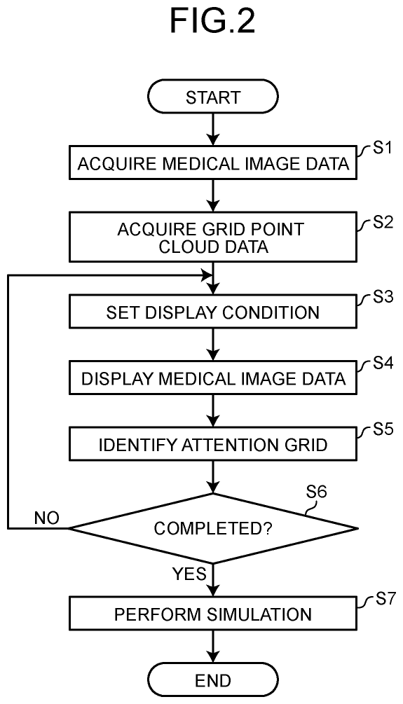

In the above, a configuration example of the medical information processing system 1 that includes the medical information processing apparatus 20 has been described. With this configuration, the processing circuitry 25 included in the medical information processing apparatus 20 easily identifies the attention grid that is used to perform the physical simulation. In the following, a process performed by the processing circuitry 25 will be described with reference to the flowchart illustrated in FIG. 2. FIG. 2 is a flowchart illustrating one example of the process performed by the processing circuitry 25 included in the medical information processing apparatus 20 according to the embodiment.

First, the image data acquisition function 25b acquires the medical image data that includes the target organ (Step S1). The image data acquisition function 25b receives the medical image data that has been captured by the medical image diagnostic apparatus 10 via the network NW, and causes the memory 24 to store the received medical image data. Here, the image data acquisition function 25b may directly acquire the medical image data from the medical image diagnostic apparatus 10, or may acquire the medical image data via the other device, such as the image storage apparatus 30.

The medical image data acquired by the image data acquisition function 25b may be any type of image as long as a target organ is included in an imaging range, and in which shape information on the target organ is stored. For example, as the medical image data that includes the target organ, the image data acquisition function 25b is able to acquire, X-ray image data, CT image data, ultrasound image data, MRI image data, PET image data, SPECT image data, or the like. Furthermore, the medical image data that includes the target organ may be a three-dimensional image or may be a two-dimensional image. In addition, as the medical image data that includes the target organ, the image data acquisition function 25b may acquire a plurality of two-dimensional images (three-dimensional images) that are obtained by capturing a plurality of time series two-dimensional image multiple times in the time direction. Furthermore, as the medical image data that includes the target organ, the image data acquisition function 25b may acquire a plurality of time series three-dimensional image (four-dimensional images) that are obtained by capturing a three-dimensional image multiple times in the time direction.

As one example, the image data acquisition function 25b acquires the medical image data when, as a trigger, an instruction is received from the user by way of the input interface 22. Alternatively, the image data acquisition function 25b may monitor the image storage apparatus 30 and acquire when, as a trigger, new medical image data is stored in the image storage apparatus 30, the new stored medical image data. Alternatively, the image data acquisition function 25b may determine whether nor not the medical image data that is newly stored in the image storage apparatus 30 satisfies a predetermined condition, and, in the case where the subject medical image data satisfies the predetermined condition, the image data acquisition function 25b may acquire the newly stored medical image data. For example, the image data acquisition function 25b may acquire when, as a trigger, the medical image data that includes a predetermined organ is newly stored in the image storage apparatus 30, the subject medical image data.

Furthermore, in the explanation described below with reference to FIG. 2, a case in which CT image data that is a three-dimensional image is acquired as the medical image data will be described. In addition, in the explanation described below with reference to FIG. 2, as one example, a case in which a patient with valvular disease of a mitral valve is a subject and a process is performed on the mitral valve of the subject as the target organ will be described. In this case, the image data acquisition function 25b acquires CT image data that includes the mitral valve of the subject at Step S1. Furthermore, at Step S2 to Step S7, a case will be described as an example in which a simulation is performed on the shape information on the mitral valve at the time of post-treatment of a percutaneous mitral valve clip technique (also referred to as MitraClip) and hemodynamic status information, on the basis of the shape information that is related to the mitral valve at the time of pre-treatment and that is obtained from the CT image. Of course, the embodiment is not limited to this, various modification are possible for the type of the medical image data, the target organ, the purpose of the simulation, and the like.

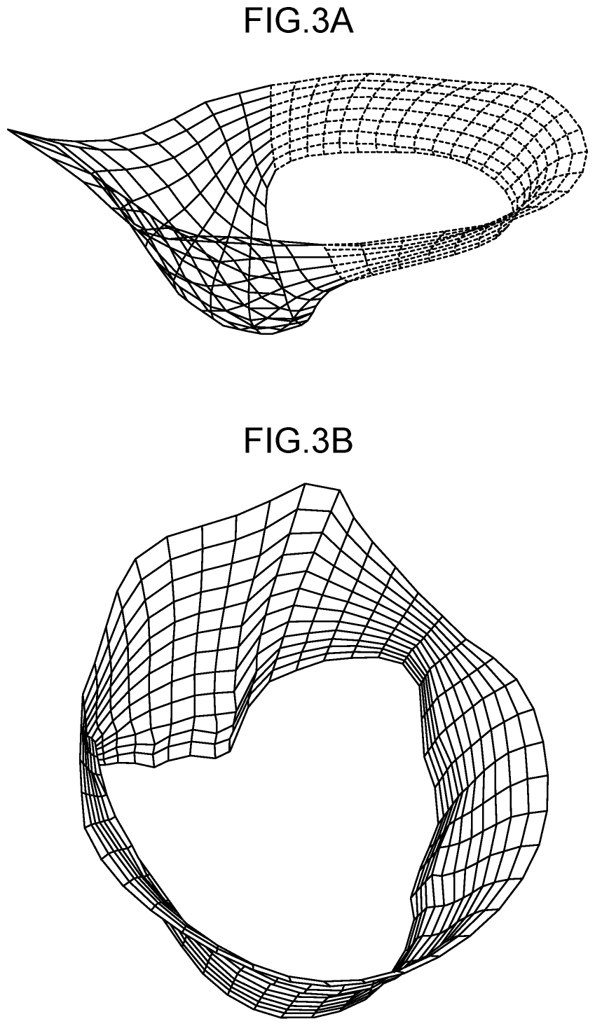

Then, the grid point cloud data acquisition function 25c acquires the grid point cloud data that is related to the target organ and that is associated with the medical image data that has been acquired at Step S1 (Step S2). The grid point cloud data is data that includes, for example, the position coordinates of each of a plurality of grid points. The grid point cloud data may be data on only the position coordinates of each of the plurality of grid points, or may be a three-dimensional image in which the plurality of grid points are arranged in a three-dimensional space. Examples of this sort of three-dimensional image include data in which the position coordinates of each of the plurality of grid points are associated with the CT image data, a mesh in which adjacent grid points are connected by a straight line or a curved line, and the like. One example of the grid point cloud data is illustrated in FIG. 3A and FIG. 3B. In FIG. 3A and FIG. 3B, the grid point cloud data is illustrated in the form of a mesh.

A method of generating the grid point cloud data is not particularly limited. A one example, it is possible to generate the grid point cloud data from the medical image data that has been acquired at Step S1. Specifically, the grid point cloud data acquisition function 25c is able to generate the grid point cloud data by identifying, from the CT image data, a mitral valve area that indicates an anatomical structure of the mitral valve, and using an already-existing technology from the identified mitral valve area. For example, the grid point cloud data acquisition function 25c generates the grid point cloud data by generating a volume rendering (VR) image from the mitral valve area included in the CT image data, and arranging the grid points on the VR image at a regular interval.

For example, the grid point cloud data acquisition function 25c identifies the mitral valve area by acquiring coordinates information on pixels that indicate the mitral valve on the CT image data. As one example, the display control function 25d causes the display 23 to display a display target image, such as a multi planar reconstruction (MPR) image, based on the CT image data. Then, the grid point cloud data acquisition function 25c identifies the mitral valve area by receiving, via the input interface 22, an input operation of specifying the position of the mitral valve area from the user who has referred to the display that is displayed on the display 23. In other words, a process of identifying the mitral valve area may be manually performed.

As another example, the grid point cloud data acquisition function 25c may identify the mitral valve area by using a known area extraction technology on the basis of the anatomical structure that is extracted to the CT image data. Examples of the known area extraction technology include a discriminant analysis method based on pixel values, such as CT values (also referred to as an Otsu's method), an area expansion method, a snake method, a graph cut method, a mean shift method, and the like.

In addition, the grid point cloud data acquisition function 25c is able to identify the mitral valve area by using an arbitrary method. For example, the grid point cloud data acquisition function 25c is also able to identify the mitral valve area by using a machine learning technology, such as a deep learning technology. For example, the grid point cloud data acquisition function 25c may identify the mitral valve area by using a shape model of the mitral valve area generated on the basis of learning data that has been prepared in advance.

As described above, in the case where the grid point cloud data has been acquired on the basis of the medical image data, the positional relationship between the grid point cloud data with respect to the medical image data is known, so that the grid point cloud data acquisition function 25c is able to associate the medical image data with the grid point cloud data. Alternatively, the grid point cloud data acquisition function 25c may generate the grid point cloud data that has already been associated with the medical image data.

In the above, an example in which the grid point cloud data is acquired on the basis of the medical image data has been described, but the embodiment is not limited to this. For example, the grid point cloud data acquisition function 25c may deform the mitral valve model indicating a general shape of the mitral valve in accordance with the information (age, a disease type, etc.) on the subject, and then generate the grid point cloud data from the deformed mitral valve model. Furthermore, for example, the grid point cloud data acquisition function 25c may deform the mitral valve model on the basis of the medical image data that has been acquired at Step S1, and then generate the grid point cloud data from the deformed mitral valve model. In this case, the grid point cloud data acquisition function 25c is able to associate the medical image data with the grid point cloud data by using an arbitrary method, that is, for example, a pattern matching method or the like.

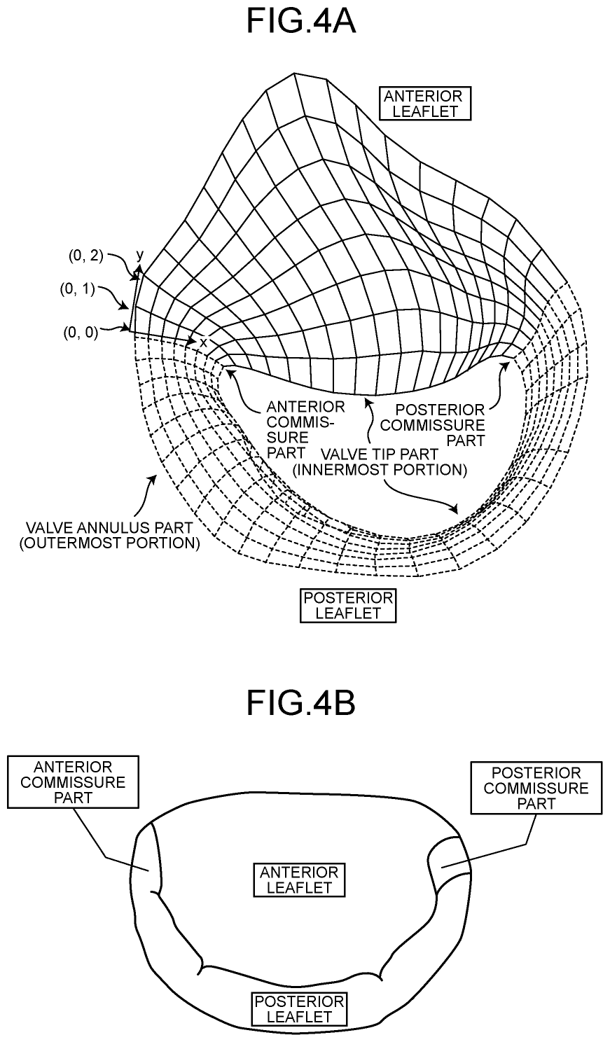

One example of the grid point cloud data related to the mitral valve is illustrated in FIG. 4A. In addition, a structure of a general mitral valve is illustrated in FIG. 4B. In FIG. 4A, an anterior leaflet area corresponding to an anterior leaflet of the mitral valve is indicated by a grid point cloud with 19 columns and 9 rows, whereas a posterior leaflet area corresponding to a posterior leaflet of the mitral valve is indicates by a grid point cloud with 25 columns and 9 rows. Of course, FIG. 4A is one example, a specific configuration (the number of grid points, placement, an array, etc.) of the grid point cloud data is not particularly limited, and the configuration of the grid point cloud data may be changed as appropriate.

In FIG. 4A, an identifier (x, y) is assigned to each of the grid points by using a portion that is a boundary between the anterior leaflet area and the posterior leaflet area and that corresponds to one end of the row wise direction as the origin, the coordinates in a row wise direction is denoted by “x”, and the coordinates in a column wise direction is denoted by “y”. In this case, an identifier (8, 0) indicates an anterior commissure part, whereas an identifier (8, 18) indicates a posterior commissure part. Furthermore, an outermost region located at a position between the anterior leaflet area and the posterior leaflet area (a position in which the x coordinates corresponds to “0” in FIG. 4A) is denoted by a valve annulus part. Moreover, an innermost region located at a position between the anterior leaflet area and the posterior leaflet area (a position in which the x coordinates corresponds to “8” in FIG. 4A) is denoted by a valve tip part.

Then, the display control function 25d sets a display condition (Step S3), and displays the medical image data under the display condition that has been set (Step S4). Examples of the display condition includes a condition related to a display range, such as the center position or an angle of the image to be displayed, and a condition related to a display color of a window level (WL) and a window width (WW).

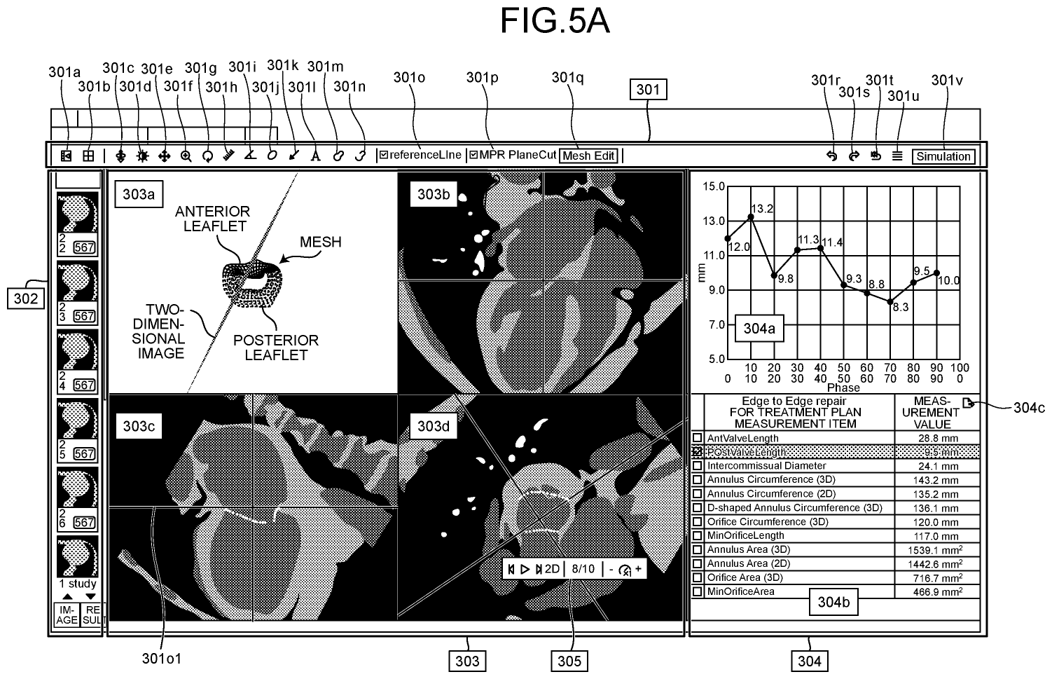

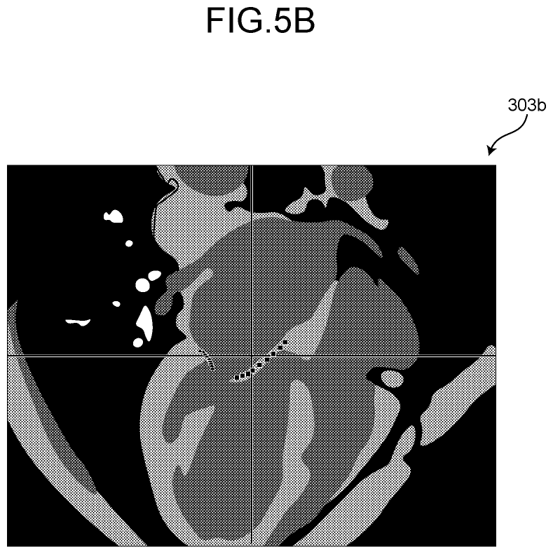

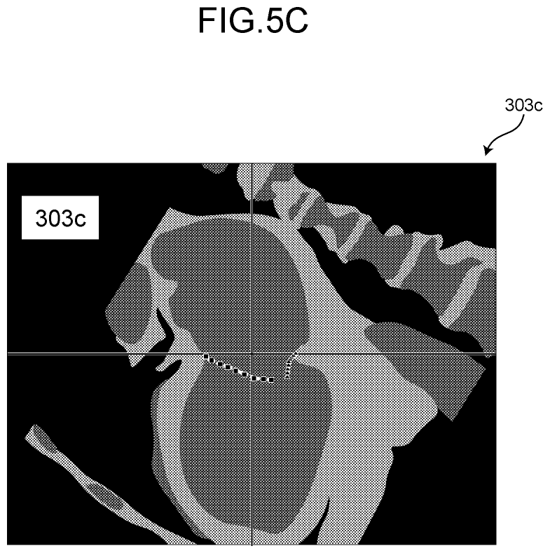

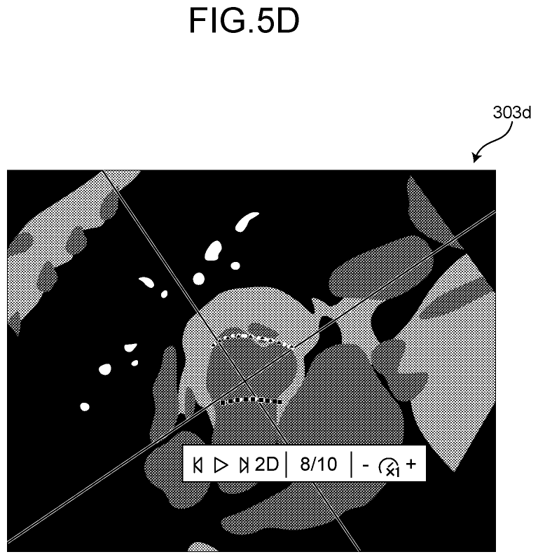

Setting of the display condition and a display example of the medical image data will be described by using FIG. 5A. FIG. 5A is a display screen that is displayed on the display 23 under the control of, for example, the display control function 25d. The display screen illustrated in FIG. 5A is just one example, and various kinds of functions that will be described later may be changed and omitted, as appropriate.

An area 301 illustrated in FIG. 5A is a menu bar in which icons and buttons corresponding to the various functions are arranged. The user is able to activate each of the functions by operating the icon arranged in the area 301 by using the input interface 22, such as a mouse.

An icon 301a is a button that is used to switch between showing and hiding an area 302. That is, as a result of the icon 301a being selected, the display control function 25d switches between showing and hiding the area 302 in which thumbnail images are displayed. For example, if the icon 301a is pressed in a state in which the area 302 is being displayed, the display control function 25d hides the area 302. Here, the display control function 25d may enlarge an area 303 or an area 304 in accordance with the size of the area 302 that becomes in a hidden state.

An icon 301b is a button that is used to change a display mode of the area 303. For example, the display control function 25d changes the number of divisions of the area 303 in accordance with the operation performed on the icon 301b. For example, in FIG. 5A, the four image display areas (301a to 301d) with two rows and two columns are set in the area 303. The display control function 25d is able to change the number of rows or the number of columns of the image display area included in the area 303 in accordance with the operation performed on the icon 301b.

Furthermore, the size of each of the image display areas included in the area 303 may be configured to be able to be changed in accordance with an operation performed on the icon 301b. For example, some sets of patterns indicating the number of image display areas and the size of these image display areas are registered as presets in advance. When the icon 301b is pressed, the display control function 25d displays an interface that is used to select the set that has been registered in advance, and receives a selection operation performed with respect to the interface, thereby setting the display mode of the area 303. The display control function 25d is also able to display an interface that is used to receive registration of a new from the user.

Icons 301c to 301g are a button group of a function of allocating an operation system of the mouse. For example, as a result of each of the icons being selected, the display control function 25d performs control such that the operation system of a left click and a drag of the mouse is allocated to the operation system that corresponds to the selected icon.

For example, the icon 301c is a button that is used to allocate a browse operation system that allows the image to be continuously displayed in the slice direction to the operation system of the left click and the drag of the mouse. When the icon 301c has been selected, and also, when an operation of left click and drag has been performed by the mouse, the display control function 25d continuously switches, on the basis of the click position and/or the drag direction, the slice image that is being displayed in the clicked area that is included in the image display area in the area 303 to the slice direction in the clicked area.

The icon 301d is a button that is used to allocate the operation system that changes the display color of an image (for example, WL, WW, or the like in a case of CT image data) to the operation system of the left click and drag operation of the mouse. When the icon 301d has been selected, and further, when the operation of left click and drag has been performed by the mouse, the display control function 25d changes, on the basis of the click position and/or the drag direction, the display color of the image that is being displayed in the clicked area that is included in the image display area in the area 303.

The icon 301e is a button that is used to allocate the operation system for a parallel shift of the image to the operation system of the operation of left click and drag performed by the mouse. When the icon 301e has been selected, and also, when the operation of left click and drag has been performed by the mouse, the display control function 25d changes, on the basis of the click position and/or the drag direction, the display position of the slice image that is being displayed in the clicked area that is included in the image display area in the area 303.

The icon 301f is a button that is used to allocate the operation system that changes an enlargement percentage of the image to the operation system of the operation of left click and drag performed by the mouse. When the icon 301f has been selected, and also, when the operation of left click and drag has been performed by the mouse, the display control function 25d changes, on the basis of the click position and/or the drag direction, the enlargement percentage of the slice image that is being displayed in the clicked area that is included in the image display area in the area 303.

The icon 301g is a button that is used to allocate an operation system that rotates an image to the operation system of the operation of left click and drag performed by the mouse. When the icon 301f has been selected, and also, when the operation of left click and drag has been performed by the mouse, the display control function 25d changes, on the basis of the click position and/or the drag direction, a display angle (an upward direction, a downward direction, or the like on the screen) of the slice image that is being displayed in the clicked area that is included in the image display area in the area 303.

Furthermore, the operations of allocating the above described functions are not limited to the operation system of the operation of left click and drag performed by the mouse. For example, the above described functions may be allocated to an operation system of an operation of right click and drag, an operation system of an operation of mouse wheel click and drag, of an operation system of a simultaneous operation of right and left click together with drag.

In addition, it may be possible to set a speed or an amount of a slice feed at the time of a browse operation, an amount of change in an enlargement percentage, an amount of movement of a parallel shift, an amount of change in a display color, and amount of rotation with respect to an amount of movement of the mouse (an amount of drag operation). Furthermore, it may be possible to change an allocation in accordance with the operation of the mouse performed at the time of selection of the subject icon. For example, control may be performed such that, when the subject icon has been selected by a left click, the operation system corresponding to the subject icon is allocated to the operation system of the operation of left click; when the subject icon has been selected by a right click, the operation system corresponding to the subject icon is allocated to the operation system of the operation of right click; when the subject icon has been selected by a simultaneous right and left click, the operation system corresponding to the subject icon is allocated to the operation system of the operation of simultaneous right and left click; and, when the subject icon has been selected by a mouse wheel click, the operation system corresponding to the subject icon is allocated to the operation system of the operation of mouse wheel click.

Icons 301h to 301n are icons that are allocated to a drawing and measurement function for various kinds of diagrams, and, the display control function 25d performs control to enable the drawing and measurement function of the various kinds of diagrams as a result of the subject icon being selected.

The icon 301h indicates a ruler function. As a result of the icon 301h being selected, for example, a left click performed by using the mouse is allocated to the ruler function. As a result of two points located in the image display area being selected by a left click, the ruler function performs a function of calculating a distance between the selected two points and displaying the calculated distance. For example, when two points located in the image display area have been selected, the display control function 25d draws a straight line on the image, measures the length of the straight line, and displays the measurement result. Furthermore, the display mode, such as the positions of the starting point and the end point of the straight line, a color of the straight line, a thickness of the straight line, and a font of a measurement value, may be adjusted by a user operation. The distance calculated by the ruler function may be a distance in a real space calculated on the basis of the enlargement percentage, a distance on the screen, or the number of pixels that are present between these two points.

The icon 301i indicates an angle calculation function. As a result of the icon 301i being selected, for example, a left click performed by using the mouse is allocated to the angle calculation function. As a result of three points that are located in the image display area by the left click being selected, the angle calculation function performs a function of calculating an angle of an acute angle or an obtuse angle that is formed by these three points and displaying the calculation result. The number of angles formed by these three points are three at a maximum, but it may be possible to calculate the angle of the acute angle or the obtuse angle at all of the positions, or it may be possible to determine a position that is used to calculate an angle on the basis of the order in which each of the points are set. For example, it may be possible to calculate an angle of an acute angle or an obtuse angle at the position of the second point. For example, when three points located in the image display area have been selected, the display control function 25d draws two straight lines on the image, calculates an angle of an acute angle or an obtuse angle formed by these two straight lines, and displays the measurement result. Furthermore, it may be possible to adjust, by a user operation, the display mode, such as the position of the starting point and the end point of each of the two straight lines, the color of each of the two straight lines, the thickness of each of the two straight lines, and the font of each of the measurement values.

The icon 301j indicates an elliptical shape display function. As a result of the icon 301j being selected, for example, a left click performed by using the mouse is allocated to the elliptical shape display function. As a result of two points in the image display area being selected by the left click, the elliptical shape display function performs a function of drawing an ellipse in which these two points are focal points. Furthermore, the elliptical shape display function is a function of calculating a circumferential length of the drawn ellipse, an internal area, and an amount of statistics of (an average value, the maximum value, the minimum value, etc.) of the pixel value in an inner part. In addition, any method may be used for a method of drawing the ellipse. For example, the ellipse may be drawn by specifying the center of the ellipse and then setting the long axis and the minor axis and the short axis. In addition, it may be possible to adjust the display mode, such as the center position, the major axis, the minor axis, the color, and the thickness of the ellipse, the font of the measurement values, by the user operation.

The icon 301k indicates an arrow display function. As a result of the icon 301k being selected, for example, a left click performed by using the mouse is allocated to the arrow display function. As a result of two points located in the image display area being selected by the left click, the arrow display function performs a function of setting the starting point and the end point of an arrow, and displaying an arrow formed by combining a straight line that connects between the starting point and the end point and a mark that indicates a direction of the starting point to the end point. It may be possible to adjust the display mode, such as the positions of the starting point and the end point of the arrow, a color of the arrow, a thickness of the arrow, and the form of a tip end part, by the user operation.

The icon 301l indicates a character string display function. As a result of the icon 301l being selected, for example, a left click performed by using the mouse is allocated to the character string display function. As a result of a single point located in the image display area being selected by the left click, the character string display function sets an area in which a character string is to be set around the single point and displays, on the area, the character string corresponding to the operation performed by the user by using the input interface 22 (a keyboard, etc.). Furthermore, it may be possible to provide a function such that a condition, such as the font, the size, and the color of the character string can be displayed. Moreover, it may be possible to adjust the display mode, such as the position of the character string to be displayed, the font of the character string, the font size, the color of the font, and the color of the background, by the user operation.

The icon 301m indicates a closed curved line drawing function. As a result of the icon 301m being selected, for example, a left click performed by using the mouse is allocated to the closed curved line drawing function. As a result of an arbitrary number of points (a point cloud) located in the image display area being selected by, for example, a left click, the closed curved line drawing function performs a function of calculating and drawing a closed curved line that passes through the point cloud. A known method can be used for the method of calculating the closed curved line from the point cloud. For example, by using a spline interpolation process, it is possible to calculate the closed curved line from the point cloud. Furthermore, the closed curved line drawing function is a function that calculates a circumferential length of the drawn closed curved line, an area in an inner part of the closed curved line, and an amount of statistics of the pixel values (an average value, the maximum value, the minimum value, etc.) in the inner part, and is a function that displays the calculation result. It is possible to adjust a display mode, such as the center position of the closed curved line, the color of the closed curved line, the thickness of the closed curved line, and the font of the measurement value, by the user operation. In addition, the closed curved line drawing function may be configured such that a shape that is determined in advance (circle, ellipse, rectangle, square, triangle, etc.) can be set so as to be able to adjust the length of each side of the corresponding shape, the angle formed by two sides, the diameter, the major axis, the minor axis and the like are adjustable, or so as to be able to draw a shape in a free form.

The icon 301n indicates an open curved line drawing function. As a result of the icon 301n being selected, for example, a left click performed by using the mouse is allocated to the open curved line drawing function. As a result of an arbitrary number of points (a point cloud) located in the image display area being selected by, for example, a left click, the open curved line drawing function performs a function of calculating and drawing the open curved line that passes through the point cloud. A known method can be used for the method of calculating the open curved line from the point cloud. Furthermore, it is possible to adjust a display mode, such as the center position of the open curved line, the color, the thickness, and the font of the measurement value, by the user operation. Moreover, the open curved line drawing function is a function of calculating an amount of statistics (a circumferential length, an area, etc.) related to the drawn open curved line, and displaying the calculation result. In addition, the open curved line drawing function may be configured such that a three-dimensional diagram (sphere, ellipsoid sphere, cuboid, triangular pyramid, etc.) can be allowed to be set so as to be able to calculate and display a surface area or a volume of the diagram, or so as to be able to draw a shape in a free form.

An icon 301o indicates a reference line display function. For example, by left clicking a checkbox that is included in the icon 301o and checking or cancelling the clicked checkbox, the icon 301o switches between showing and hiding the line (reference line) that indicates the position corresponding to the cross section that is displayed in another area, in an area (for example, in FIG. 5A, an area 303b, an area 303c, and an area 303d) to be targeted in the image display area. For example, a reference line 301o1 indicated in the area 303c illustrated in FIG. 5A indicates a cross-sectional position of the slice image that is being displayed in the area 303d. Regarding the reference line, it may be possible to change the display position of the reference line and intersection positions of a plurality of reference lines in the image display area on the basis of an instruction received from the user. At this time, the cross-sectional position of the image that is being displayed on the corresponding image display area is changed to the position corresponding to the changed reference line.

An icon 301p indicates a function of displaying the two-dimensional image by being superimposed on the three-dimensional image. Here, the three-dimensional image may be a rendering image, such as a VR image or a surface rendering (SR) image, or may be grid point cloud data that is generated in a three-dimensional space. This sort of three-dimensional grid point cloud data is generated at Step S2 as described above. In FIG. 5A, as an example of the three-dimensional grid point cloud data, a mesh related to a mitral valve is illustrated in an area 303a.

More specifically, when the checkbox included in the icon 301p has been checked by a left click, the display control function 25d displays the three-dimensional image, such as the mesh, by associating the two-dimensional image with the three-dimensional position. For example, the display control function 25d identifies the position of the two-dimensional image with respect to the three-dimensional mesh on the basis of the positional relationship between the position of the three-dimensional mesh in the CT image data (volume data) and the position of the two-dimensional image in the CT image data. Then, the display control function 25d causes a superimposed image indicated in the area 303a illustrated in FIG. 5A by arranging the two-dimensional image at the identified position of the three-dimensional mesh.

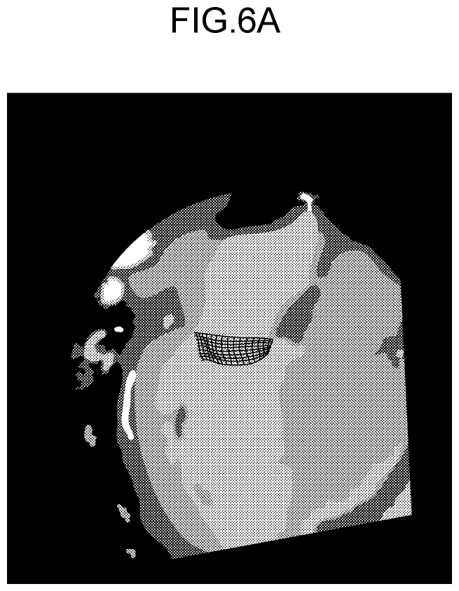

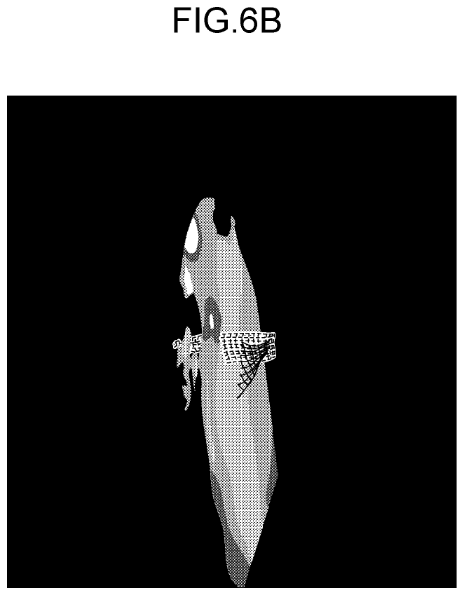

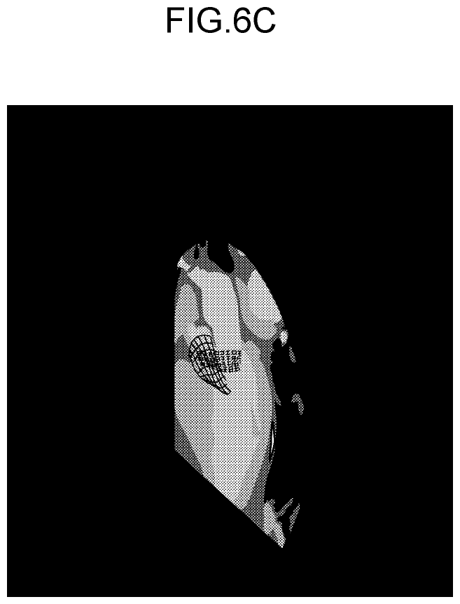

When the images are superimposed, as illustrated in FIG. 6A, the display control function 25d may perform control such that the mesh that is located closer to the near side than the two-dimensional image with respect to the observation direction is displayed, and perform control such that the mesh that is located further away from the two-dimensional image is not displayed. Furthermore, as illustrated in FIG. 6B and FIG. 6C, the display angle of the mesh and the two-dimensional image may be configured to be rotatable as appropriate. For example, when a left click and drag operation have been performed by using the mouse in the area 303a, the display control function 25d rotates, on the basis of the click position and/or the drag direction, the display angle of the mesh and the two-dimensional image that are displayed in the area 303a. Furthermore, when the checkbox included in the icon 301p has been left clicked and the check has been cancelled, the two-dimensional image becomes in a hidden state.

The two-dimensional image (the two-dimensional image displayed in the area 303a illustrated in FIG. 5A) that is displayed by being superimposed on the three-dimensional image is selected from among the images that are displayed in, for example, the image display areas (the areas 303b to 303d) that are included in the area 303. The two-dimensional image that is displayed by being superimposed on the three-dimensional image may be the three images that are displayed in the areas 303b to 303d, or may be one or two images that are selected by the user. For example, when a right click is performed in one of the areas 303b to 303d, a context menu is displayed, and the two-dimensional image that is to be displayed by being superimposed on the three-dimensional image is selected in accordance with the operation performed on the context menu by the user. Furthermore, it may be possible to determine in advance the area, in which the two-dimensional image to be displayed by being superimposed on the three-dimensional image is displayed, from among the areas 303b to 303d.

An icon 301q indicates a mesh editing function. As a result of the icon 301q being selected, for example, it is possible to edit the mesh that is being displayed in the area 303a. In other words, with the mesh editing function, it is possible to edit the grid point cloud data that has been generated at Step S2 described above. Furthermore, in the case where the icon 301q is not selected, the mesh editing function does not work.

For example, in FIG. 5A, the entire image of the mesh that is related to the mitral valve is displayed in the area 303a. Here, for example, as illustrated in FIG. 5B to FIG. 5D, marks that indicate intersection points with the mesh are displayed in each of the image display areas indicated by the area 303b to 303d. In other words, in FIG. 5B to FIG. 5D, the marks that indicate the intersection points between the two-dimensional image that is displayed in the image display area and the straight line or the curved line that connects the grid points of the mesh are displayed. More specifically, in FIG. 5B to FIG. 5D, in each of the image display areas corresponding to the area 303b, the area 303c, and the area 303d, each of the intersection points with a portion that corresponds to the anterior leaflet out of the entire mesh is indicated by a square mark, whereas each of the intersection points with a portion corresponding to the posterior leaflet is indicated by a triangular mark.

For example, the mesh is constituted by the plurality of grid points and a plurality of straight lines each of which connects adjacent grid points. The display control function 25d obtains a cross section at a cross-sectional position of the image that is displayed in each of the image display areas corresponding to the areas 303b to 303d related to the plurality of straight lines constituting the mesh. For example, as illustrated in FIG. 4A, in a case of a mesh in which the anterior leaflet area is indicated by the grid point cloud with 19 columns and 9 rows and the posterior leaflet area is indicated by the grid point cloud with 25 columns and 9 rows, the cross sectional surface thereof is represented by 18 marks (nine square marks indicating anterior leaflet area in cross section and nine triangular marks indicating the posterior leaflet area in cross section illustrated in FIG. 5B to FIG. 5D) at the maximum. That is, in a case of a mesh having a grid point cloud with 18 rows, 18 marks are displayed in the case where the two-dimensional image is arranged so as to intersect with all of the 18 marks, one to 17 marks are displayed in the case where the two-dimensional image is arranged so as to intersect with only a part of the 18 rows, a mark is not displayed in the case where the two-dimensional image is arranged so as to intersect with of the 18 rows.

For example, by moving the cross sectional surface of the mesh displayed in each of the image display area corresponding to the areas 303b to 303d by a left click and a drag, the user is able to modify the shape of the mesh in accordance with an amount of the movement. Furthermore, it may be possible to adjust the display mode, such as the shape of the mark, the color of the mark, and the number of marks, that indicates the cross sectional surface of the mesh illustrated in FIG. 5B to FIG. 5D by the user operation.

A description will be given here by referring back to FIG. 5A. An icon 301r indicates an Undo (cancel) function. As a result of the icon 301r being selected, a display that is displayed in the area 303 returns to the state before the last operation is performed. For example, the display control function 25d is able to implement this function by storing the display condition in the area 303 every time an operation is performed. Furthermore, in addition to returning to the state of the last operation, the display control function 25d may store a predetermined number of display conditions after the past operations, exhibit the plurality of display conditions to the user, and return, as a result of the user specifying an arbitrary display condition, the state to the state at the time of the display condition that has been specified by the user. At this time, the display control function 25d may store the display condition in time series every time a single operation is performed, or may store the display condition only when an operation that satisfies a specific condition. For example, the display control function 25d may store the display condition only when an operation of changing a display mode of a specific image display area (for example, the area 303b) has been performed. This sort of specific condition is set in advance.

An icon 301s indicates a Redo (try again) function. By selecting the icon 301s when the display that is displayed in the area 303 is returned to the state before the last operation by using the Undo function of the icon 301r, the redo function cancels the operation performed by the Undo function and returns to the state before the Undo function is performed.

An icon 301t indicates a reset function. As a result of the icon 301t being selected, the display condition in the area 303 returns to the predetermined condition. Any condition may be used for the predetermined condition, and, as one example, a condition at the time of activation may be used. In other words, when the function corresponding to the display control function 25d of displaying the display screen illustrated in FIG. 5A has been activated, first, a display is performed under the condition to be set in advance, and then, the display is variously changed in accordance with the operation received from the user. When the icon 301t has been selected, the display control function 25d returns the display that is displayed under the predetermined condition at the time of activation.

An icon 301u is a button that is used to display a setting screen for setting a display condition of the area for a superimposed display with respect to a two-dimensional image, such as a rendering image including a VR image or a SR image, and an MPR image. Specifically, for example, at Step S2, positional information on the anatomical structures (each of the valve leaflets, each atrium, each cardiac ventricle, calcification, etc.) that are included in the medical image data that has been acquired at Step S1 is identified. When the area that indicates each of the various kinds of anatomical structures is displayed by being superimposed on the VR image and the MPR image, if, for example, the user selects the icon 301u by a mouse operation, the setting screen for setting the display condition of the area (area indicating the anatomical structure) that is to be displayed by being superimposed on the VR image and the MPR image is displayed.

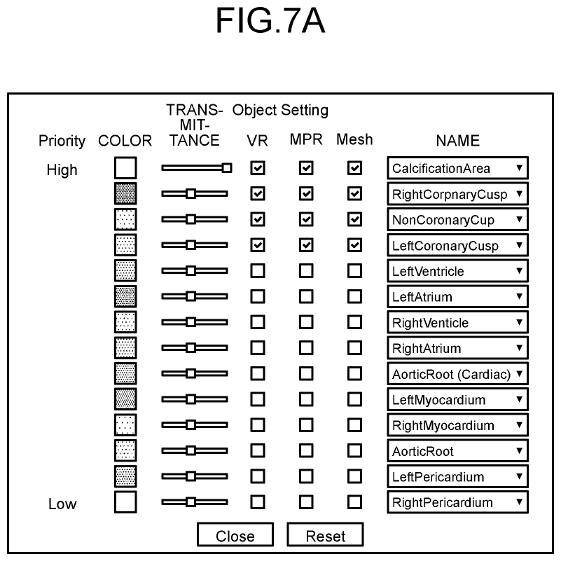

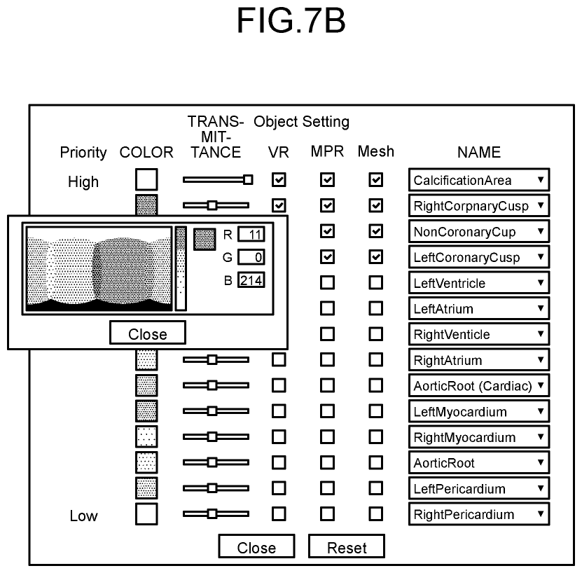

FIG. 7A and the FIG. 7B are diagrams each indicating one example of the setting screen of the display condition according to the embodiment. For example, as illustrated in FIG. 7A, the setting screen includes setting items that are related to “Priority”, “color”, “transmittance”, “VR”, “MPR”, “Mesh”, and “name”.

In the item of “Priority”, a display priority order of the area to be specified (to be specified from the combo box of “name” disposed on the right side) is set. For example, the item of “Priority” indicates that the display priority order is higher for the area that is specified on the setting screen, and, in the case where a plurality of areas correspond to the same coordinates in the image, an area with high priority is displayed.

In the item of “color”, a color that is allocated at the time of superimposed display performed on the VR image and the MPR image with respect to the corresponding area (specified from the combo box of “name” disposed on the right side) is set. For example, the item of “color”, a sample of the color is displayed. For example, if the user selects the area that indicates a sample of the color, the display control function 25d displays, as illustrated in FIG. 7B, a color map and an input box of the values. The user is able to allocate an arbitrary color to the target area by selecting a color from the color map or by inputting the RGB values.

In the item of “transmittance”, a transmittance at the time of a superimposed display performed on the VR image and the MPR image with respect to the corresponding area (specified from the combo box of “area name”). For example, “transmittance” can be set by a slider bar at an interval of 1% between 0 to 99%, and in a case of 0%, a superimposed display is performed in a state in which no image is transmitted (i.e., a background image is invisible). Furthermore, although not illustrated in FIG. 7A and FIG. 7B, it may be possible to construct the setting screen such that a display condition of a color saturation, a brightness, or the like can be set, or, a texture can be set instead of the color. Moreover, it may be possible to construct the setting screen such that all of the transmittance together can be set by selecting a “link” checkbox (not illustrated). More specifically, when the “link” checkbox has been selected, it may be possible to perform control such that all of the pieces of transmittance are set to the same value, or it may be possible to perform control such that the transmittance is increased or decreased overall while maintaining the relationship among the pieces of transmittance corresponding to the respective areas at the time of the selection of the “link” checkbox.

The item of “VR” is a checkbox for specifying the area that is to be displayed on the VR image. Furthermore, the “MPR” is a checkbox for specifying the area that is displayed on the MPR image. Moreover, although not illustrated in FIG. 7A and FIG. 7B, it may further provide a button that allows a check or a cancellation of the check to be performed at the same time with respect to the checkboxes of the “VR” and the “MPR” or all of the checkboxes of the “Mesh”.

The item of “Mesh” is a checkbox for specifying whether the display mode of the area in which a superimposed display is performed on the VR image and the MPR image is in a mask format or a mesh format. Specifically, in a case of the mesh format, the display control function 25d displays the mesh that has been acquired at Step S2, as indicated by the area 303a illustrated in, for example, FIG. 5A. In contrast, in a case of the mask format, the display control function 25d displays the image, such as a VR image, an SR image, or an MPR image. Furthermore, if the mesh format is used, even when the “transmittance” is 0%, a background image is able to be viewed through a gap in the mesh. In contrast, if the mask format is used and “transmittance” is 0%, the background image is not viewed.

In the item of “name”, an area that is displayed on the basis of the set priority order or the display condition. For example, the user specifies the area by the combo box that is arranged in the column of “name”. Furthermore, it may be possible to perform control such that the same area is not set in the plurality of combo boxes. For example, in the case where an area that has already been set by another combo box is specified to a certain combo box, it may possible to perform control such that the subject area is not able to be specified or setting of an already exist combo box is canceled. Alternatively, it may be possible to perform control such that priority is given to an area that has a higher priority of setting while enabling to set the same area into the plurality of combo boxes. Furthermore, in FIG. 7A, the setting is configured to display a calcification area, a left coronary cusp (LCC), a right coronary cusp (RCC), and a non coronary cusp (NCC) of an aortic valve on the VR image, the MPR image, and the mesh.

The “Close” is a button that is used to hide the setting screen, and, the “Reset” is a button that is used to return the setting state to the initial state. Furthermore, regarding the timing at which the display condition that is set by the subject setting screen is reflected to each of the areas, the set condition may be reflected immediately after each of the condition has been set, or the timing may be collectively reflected after the selection of the “Close” button.

An icon 301v is a button that is used to start a simulation mode. The simulation mode will be described later.

The area 302 displays an icon that indicates the image that satisfies the specified condition. For example, by using an interface (not illustrated), the user specifies information on the subject, such as the name, the subject ID, the date of birth, the body weight of the subject; information on the image, such as the type of the modality of the image the name of the imaging apparatus, the imaging date, the imaging condition, the reconstruction condition; and the like. The image data acquisition function 25b acquires, from the medical image diagnostic apparatus 10 or the image storage apparatus 30, the volume data that satisfies the above described condition specified by the user. For example, the image data acquisition function 25b acquires information on the specified condition from the header of digital imaging and communications in medicine (DICOM) of the image, PACS, an electronic medical record, a radiology information system (RIS), a hospital information system (HIS), or the like; compares the acquired condition with the condition that has been specified by the user; and then, acquires the volume data that satisfies the condition that has been specified by the user. Moreover, in the following, an example in which a single piece of four-dimensional CT image data of a predetermined single subject has been specified will be described, but images of a plurality of subjects, or modalities of different types (for example, CT image data and ultrasound image data, etc.) may also be specified.

The display control function 25d displays a thumbnail as an icon representing an image that satisfies, for example, the specified condition. Specifically, the display control function 25d generates thumbnail images from the volume data that has been acquired by the image data acquisition function 25b, and displays the generated thumbnail images in the area 302. For example, the display control function 25d is able to generate the thumbnail images by reducing the size of the two-dimensional image having a typical cross section included in the volume data in accordance with the size of the area 302.

In the above, thumbnail has been described as the icon that represents the image that satisfies the specified condition, but the display control function 25d is able to display various icons in the area 302 instead of or in addition to the thumbnail images. For example, the display control function 25d may display, in the area 302, a character string or a symbol that indicates the acquired volume data, or, various kinds of diagrams, images, schema images, and the like stored in the memory 24 in advance. Furthermore, the display control function 25d is able to display basic information (imaging date, the number of sliced pieces, a reconstruction function, etc.) on the volume data side by side together with the thumbnail images and the icons described above. In such a case, for example, the display control function 25d acquires these pieces of information from the DICOM header of the image, the PACS, the electronic medical record, the RIS, the HIS, or the like, and displays the information in association with the thumbnail images and the icons. Furthermore, the basic information to be displayed may be determined in advance, or the user may specify the basic information that is to be displayed.

For example, the user drags and drops the icon of the thumbnail images that are displayed in the area 302 into the area 303. In response to this operation, the display control function 25d generates an image to be displayed from the volume data corresponding to the selected thumbnail image, and displays the generated image in the area 302. Here, if an image has already been displayed in the area 303 at the time of the drag and drop operation, the display control function 25d displays a confirmation screen (not illustrated) (for example, a display that urges the user to save the image, or the like) to the user. Then, after the display control function 25d receives an operation of a positive response to the confirmation screen from the user, the display control function 25d displays the image corresponding to the dragged and dropped icon by removing the already displayed image from the area 303.

At the time of displaying the image in the area 303, the display control function 25d displays the image on the basis of the display condition that is determined in advance. Here, the display condition is an allocation of the images to be displayed in a plurality of display areas that are included in the area 303 (for example, what sort of image is to be displayed in which area from among the areas 303a to 303d illustrated in FIG. 5A), a cross-sectional position, an enlargement percentage, a WL, and a WW at the time of a display of the cross-sectional image, and the like. When the display control function 25d displays the image in the area 303, the display control function 25d acquires the above described display condition, generates an image on the basis of the acquired display condition, and displays the generated image in the area 303.

Furthermore, the above described display condition is one example, and any condition may be set. Moreover, the display condition may be arbitrarily changed by the user. In such a case, for example, the display control function 25d displays a GUI for setting a display condition, and receives the display condition specified by the user.

As described above, the area 303 is the image display area, and displays various kinds of images. For example, FIG. 5A illustrates the initial arrangement of each of the areas at the time of reading the image, and four areas denoted by the area 303a to the area 303d are set.

In FIG. 5A, in the area 303a, a three-dimensional image of the mitral valve is displayed. More specifically, in the area 303a, the three-dimensional mesh of the mitral valve that has been acquired at Step S2 is displayed. For example, in the case where the checkbox of “Mesh” illustrated in FIG. 7A and FIG. 7B has been checked, the display control function 25d displays the mesh as illustrated in FIG. 5A. In contrast, in the case where the check has been canceled, the display control function 25d displays a VR image, a SR image, or the like of the mitral valve, instead of the mesh illustrated in FIG. 5A, or, in another area (the area 303b to the area 303d).

Furthermore, in FIG. 5A, in each of the area 303b to the area 303d, the two-dimensional image of the mitral valve is displayed. For example, the display control function 25d displays, in each of the area 303b to the area 303d, the MPR image that is set on the basis of the mitral valve. As one example, the display control function 25d identifies, from the mitral valve area that has been identified at Step S2, a surface that passes through a cardiac apex portion and that is perpendicular to an annulus surface of the mitral valve, generates a three way MPR image by using the identified surface as a reference surface, and displays each of the images in the area 303b to the area 303d in an associated manner. The annulus surface of the mitral valve is, for example, a least squares plane that is calculated from the closed curved line constituted by the valve annulus part illustrated in FIG. 4.

Of course, the display illustrated in FIG. 5A is one example, various modifications are possible for the display of the area 303. For example, the display control function 25d may display, in the area 303, an image of an arbitrary cross section specified by the user or an image with an arbitrary type. As one example, the display control function 25d may generate an image with the known type, such as the VR image, the SR image, the maximum intensity projection (MIP) image, or the minimum intensity projection (MinIP) image, and displays the generated image in the area 303.

Furthermore, the display condition of the image in the area 303 is able to be changed by using the various kinds of functions that are set in the area 301 and the area 302 described above as appropriate. For example, regarding the image in the area 303, the display control function 25d is able to change an observing cross section, the slice feed (browse), the enlargement percentage, the center position (parallel shift), the WL, the WW, or the like on the basis of an instruction received from the user.

Furthermore, the display control function 25d may display, in each of the areas, the information that has been set in advance, or, the information that is specified by the user in a superimposed manner. For example, the display control function 25d displays, at a predetermined position included in each of the image display area corresponding to the area 303a to the area 303d, information on the subject, such as the name, the subject ID, the date of birth, and the body weight of the subject, information on the image, such as the type of the modality of the image, the name of the imaging apparatus, the imaging date, the imaging condition, and the reconstruction condition, or the like. For example, the display control function 25d acquires the information specified by the user from among the pieces of the above described information on the subject and the pieces of above described information on the image, from the header of the DICOM of the image, the PACS, the electronic medical record, the RIS, the HIS, or the like, and displays the acquired information in each of the image display areas corresponding to the area 303a to area 303d.

Furthermore, in FIG. 5A, in the area 303d, a controller 305 for displaying a cine display is displayed. In the controller 305, a play button, a stop button, a speed up button, a speed down button, a back button to a start image, a forward button to the last image, a forward button to a next image of a cardiac phase, a forward button to a previous one image of the cardiac phase, and the like are set, and, control is performed such that, as a result of the user specifying one of the buttons by an operation performed by using a mouse click or the like, the function allocated to each of the various kinds of buttons is performed. Moreover, the display order at the time of the cine display may be determined on the basis of the selection of the thumbnails, or may be determined on the basis of the imaging date and time obtained from the header of the DICOM or the like, or the order of the cardiac phases that are set on the basis of an R-R interval. Furthermore, in the case where the user gives some instruction to the image during the cine display (a slice feed, a parallel shift, a change in an enlargement percentage, a change in gradation, measurements obtained from various kinds of measurement functions, etc.), the subject controller may be hidden.

In FIG. 5A, the area 304 is a display area of the measurement result. For example, at Step S2, an attention area, such as a mitral valve area is identified from the CT image data, a value (measurement value) indicating the feature (measurement item) of the attention area is calculated on the basis of each of the attention areas. An area 304a is an area that is used to display the measurement results as a graph. Furthermore, an area 304b is an area that is used to display, as the measurement result, a list indicating the relationships between various kinds of the names of the measurement items and the measurement values. Example of the measurement item include a length of the anterior leaflet (AntValveLength), a length of the posterior leaflet (PostValveLength), a distance between commissures (Inter commissual Diameter), a circumferential length of the valve annulus (Annulus circumference), an area of the valve annulus (Annulus Area), a circumferential length of a D-shaped valve annulus (D-shaped Annulus circumference), a circumferential length of of the valve orifice (Orifice circumference), an area of the valve orifice (Orifice Area), a minimum circumferential length of the valve orifice (MinOrificeLength), a minimum area of the valve orifice (MinOrificeArea), and the like.

In FIG. 5A, as a display example of the area 304a, a line graph constructed by using the vertical axis as the measurement values and the horizontal axis as the cardiac phases (Phase) is illustrated. However, the graph that is displayed in the area 304a is not limited to this. For example, it may be possible to display a line graph indicating a relationship between each of a measurement value and a cross-sectional position by using the horizontal axis as a cross-sectional position of an arbitrary direction. The line graph may also display an arbitrary single valve leaflet and a plurality of measurement items. Furthermore, a plurality of graphs may be displayed in order to display the plurality of measurement items of the plurality of valve leaflets. Moreover, regarding a single measurement item, a relationship with each of the valve leaflet may be displayed in the same graph. In addition, instead of the line graph, for example, it may be possible to represent the feature of the valve leaflet in an arbitrary phase by using a radar chart.

It may be possible to perform control such that the form of the graph displayed in the area 304a is changed to the form suitable for each of the measurement items by selecting the checkbox disposed on the left side of the list that is being displayed in the area 304b. The relationship between the measurement items and the form of the graph may be set in advance. Furthermore, the display mode, such as a color and the thickness, of the graph may be set by the user, or, may be changed in accordance with the display mode, in each of the areas, of the valve leaflet that is set by the setting screen illustrated in FIG. 7A and FIG. 7B. Moreover, it may be possible to set the type of the measurement item to be displayed. In addition, it may be possible to perform control such that, when a checkbox disposed on the left side of each of the measurement items in the area 304b has been selected, a position in the image corresponding to the measurement position related to the corresponding measurement item or a cross section of the image is displayed in the area 303. When an icon 304c has been selected, the display control function 25d stores various kinds of measurement results in the memory 24. For example, when the icon 304c has been selected, the display control function 25d outputs a table that indicates the relationship between the measurement value associated with the various kinds of measurement items and the phase or the slice in the form of a file of Comma Separated Values (CSV), or the like with respect to the storage area that is included in the memory 24 and that is specified by the user.

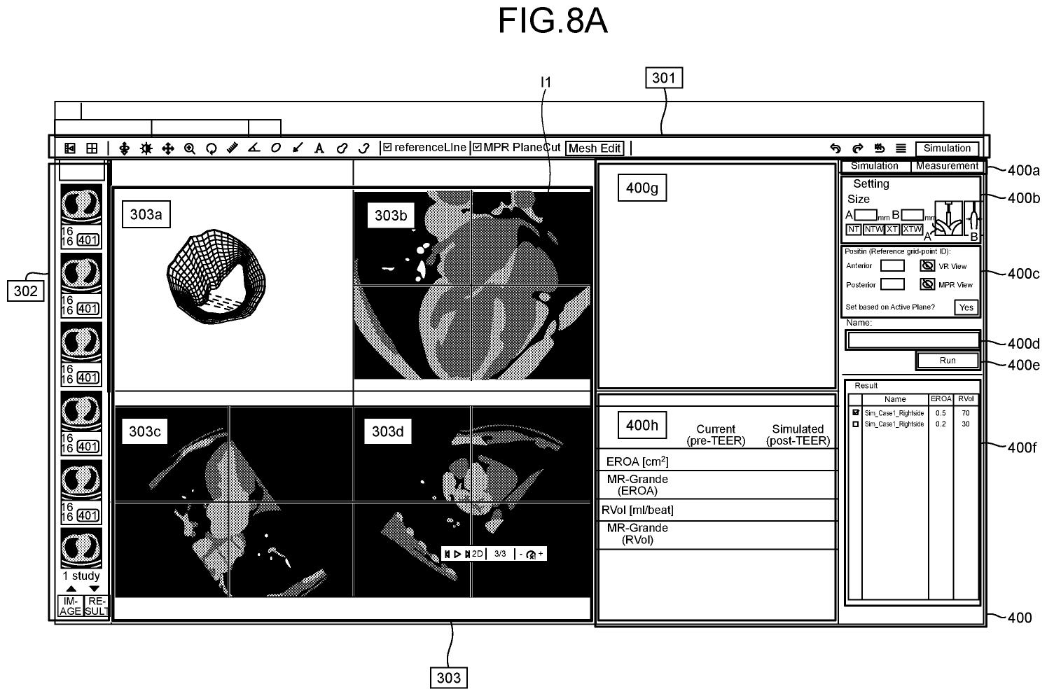

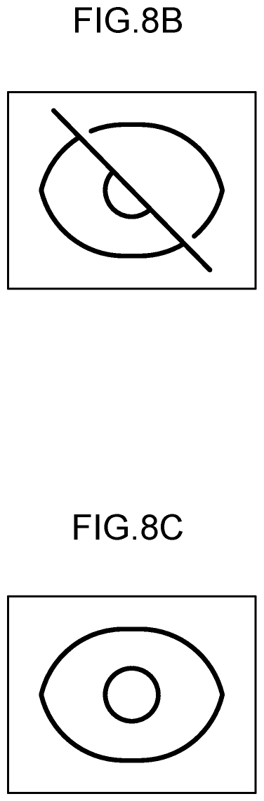

Then, the identification function 25e identifies an attention grid that is included in the grid point cloud data on the basis of the display condition of the medical image data that has been set at Step S3 (Step S5). The process performed at Step S5 is started when, as a trigger, the button of, for example, the icon 301v has been selected and the state shifts to the simulation mode. In the following, a process performed after the button of the icon 301v has been selected will be described with reference to FIG. 8A. FIG. 8A is a display example in the simulation mode.

For example, in FIG. 5A, when the icon 301v has been selected, the display control function 25d displays an area 400 illustrated in FIG. 8A instead of the area 304. In an area 400a in the area 400, two tabs of “Simulation” and “Measurement” are displayed, and, in FIG. 8, the tab of “Simulation” is selected.

Furthermore, in FIG. 5A and FIG. 8A, as one example, the area 400 is displayed by a larger size than that of the area 304. Accordingly, the size of the area 303 illustrated in FIG. 8A is smaller than the size of the area 303 illustrated in FIG. 5A. Moreover, in FIG. 8A, in the case where “Measurement” displayed in the area 400a has been selected, the display control function 25d again displays, instead of the area 400, the area 304 that includes the measurement values and the graph of the measurement values. Here, the display control function 25d may record the state of the area 304 just before “Simulation” has been selected (for example, the state of the graph, such as the measurement item or the width of the axis that are being displayed), and may display, when “Measurement” is selected, the area 304 that is in the recorded state without any change. Moreover, even if the tab is switched between “Simulation” and “Measurement”, the image display state (for example, the cross-sectional position, the enlargement percentage, the WW, the WL, the display angle, etc.) in the area 303 is not changed. If the image display state in the area 303 has been changed by a user operation in a period of time between the selection of “Simulation” and the selection of “Measurement”, it may be possible to update the measurement value and the display of the graph on the basis of the new image display state.

For example, as illustrated in the areas 303b to 303d, in the case where the plurality of images are displayed, first, the identification function 25e selects an attention image (also referred to as an Active Plane) that is used to refer to the display condition from among the plurality of displayed images. In the following, a case in which an image I1 that is being displayed in the area 303b is selected as an attention image will be described. Furthermore, the image I1 is an MPR image (cross-sectional image) obtained on the basis of the CT image data acquired at Step S1. As illustrated in FIG. 8A, the display control function 25d may highlight the image I1 that is being selected as the attention image or the area 303b in which the image I1 is being displayed by enclosing the image I1 or the area 303b by, for example, a colored frame or a thick frame that is thicker than that of the other area. The attention image may be selected on the basis of an instruction received from the user, or the image that is displayed in the image display area that has been set in advance from among the areas 303b to 303d may be automatically selected as the attention image.

The identification function 25e identifies the attention grid on the basis of the display condition of the image I1 that is the attention image. For example, the identification function 25e identifies the attention grid on the basis of the display condition related to the display range of the image I1. Examples of the display condition related to the display range include a display angle of the image I1, the center position of the image I1 (the position in the slice direction, and the position on a plane parallel to the image I1), an enlargement percentage, and the like.

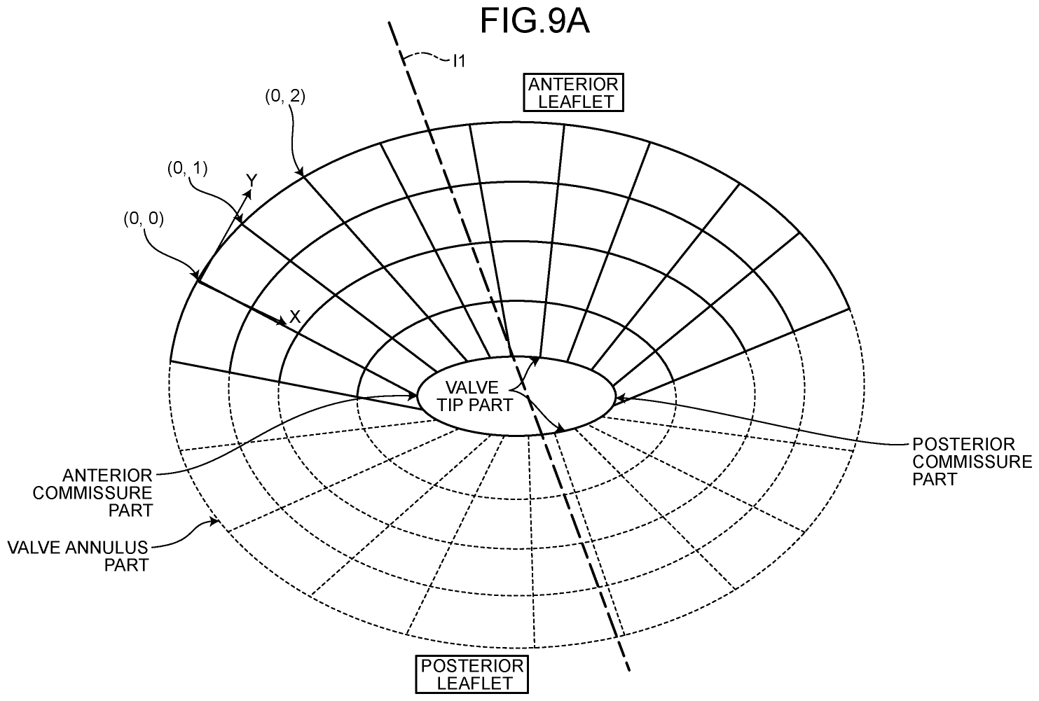

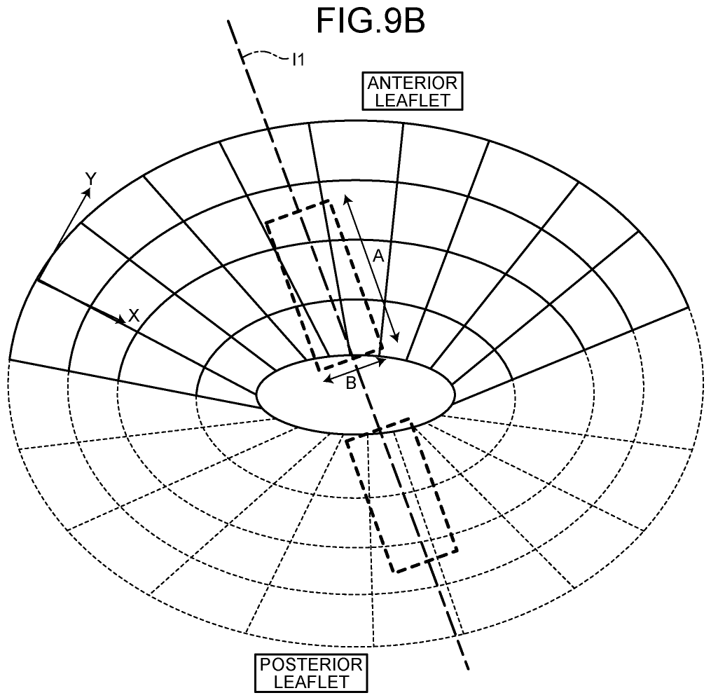

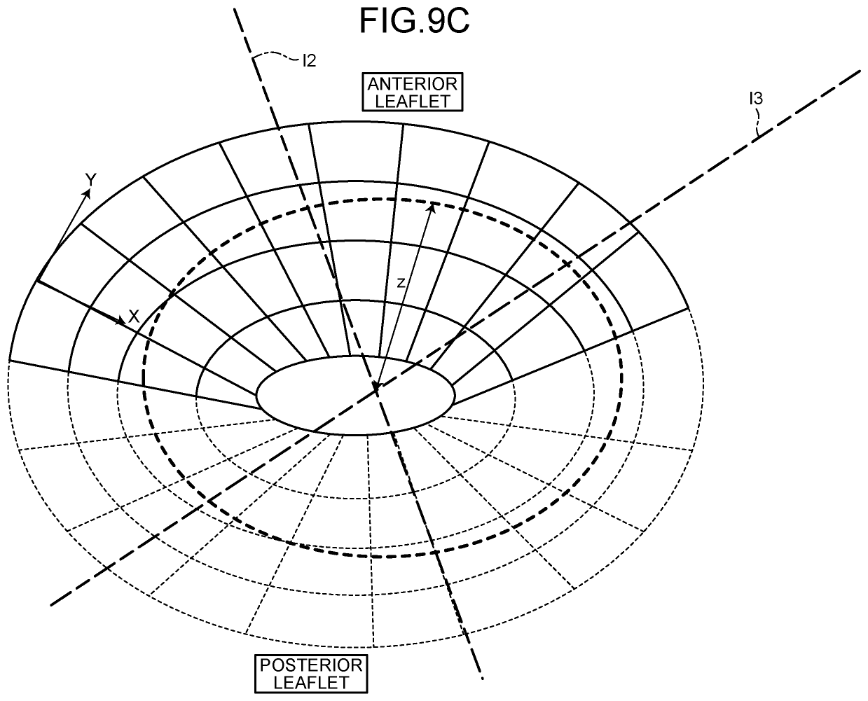

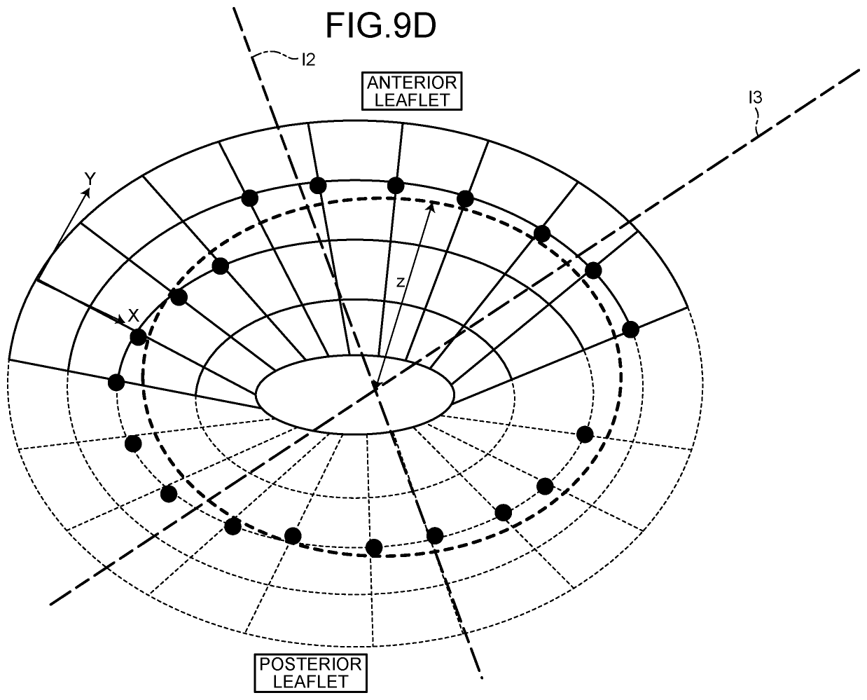

In the following, a specific explanation will be given with reference to FIG. 9A. FIG. 9A is a simplified diagram illustrating the mesh related to the mitral valve by using ellipses and straight lines for explanation. More specifically, in FIG. 9A, each of the intersection point between the ellipse and the straight line indicates a grid point. Furthermore, each of the ellipses and the straight lines illustrated in FIG. 9A correspond to a line (a straight line or a curved line) that connects the grid points. For example, each of the ellipses illustrated in FIG. 9A is a line obtained by connecting the grid points in the column wise direction, whereas each of the straight lines is a line obtained by connecting the grid points in the row wise direction. Furthermore, in FIG. 9A, the anterior leaflet area is indicated by the solid lines, whereas the posterior leaflet area is indicated by the broken lines. In the case where the mitral valve is represented in the image I1 in the area 303b, as illustrated in FIG. 9A, the cross-sectional position of the image I1 intersects with the mitral valve. Moreover, in FIG. 9A, a description will be made on the assumption that the coordinates in the row wise direction is denoted by “X”, the coordinates in the column wise direction is denoted by “Y”, an identifier (X, Y) is assigned to each of the grids.

For example, first, the identification function 25e sets the size of the treatment device. The treatment device is a clip (MitraClip device) that is placed in the mitral valve by, for example, a percutaneous mitral valve clip operation. The size of the treatment device is specified by, for example, the user. As one example, the user specifies the size of the clip by inputting the fields denoted by “A” and “B” displayed in an area 400b illustrated in FIG. 8A. For example, “A” denotes the length of a pinch portion (a portion that is brought into contact with the mitral valve at the time of placement in the mitral valve) of the clip, whereas “B” denotes the size of the pinch portion in the width direction.

The user may input each of the values of “A” and “B”, or may select one of a plurality of preset values. For example, in the area 400b illustrated in FIG. 8A, four preset values of “NT”, “NTW”, “XT”, and “XTW” are displayed. For example, in the case where “NT” has been selected by the user, the identification function 25e automatically sets the values of “A=9, and B=4”. Furthermore, in the case where “NTW” has been selected by the user, the identification function 25e automatically sets the values of “A=9, and B=6”. Moreover, in the case where “XT” has been selected by the user, the identification function 25e automatically sets the values of “A=12, and B=4”. In addition, in the case where “XTW” has been selected by the user, the identification function 25e automatically sets the values of “A=12, and B=6”.

Furthermore, a method of specifying the size of the treatment device is not particularly limited. For example, the identification function 25e may determine the size of the treatment device on the basis of the condition related to the subject, the condition related to the valve, and the like. For example, the identification function 25e is able to automatically determine the size of the treatment device on the size of the mitral valve area identified at Step S2. In this case, it may be possible to define in advance the correspondence relationship between the condition related to the subject or the condition related to the valve and the size (a type, a model number of the treatment device, or the like may be used) of the treatment device.

Then, the identification function 25e sets a placement position of the treatment device. In each of the row of the mesh related to the mitral valve illustrated in FIG. 9A, the identification function 25e sets the placement position on the basis of the range that is determined by the display condition of the image I1 corresponding to the attention image and the size of the treatment device. For example, as illustrated in FIG. 9B, the identification function 25e sets the range that is represented by the length “A” of the treatment device along the cross-sectional position of the image I1 and the width “B” of the treatment device centered at the cross-sectional position, in the direction from the valve tip part of the anterior leaflet toward the valve annulus part. Similarly to the posterior leaflet, the identification function 25e sets the range that is represented by the length “A” of the treatment device along the cross-sectional position of the image I1 and the width “B” of the treatment device, in the direction from the valve tip part of the posterior leaflet toward the valve annulus part. Furthermore, in FIG. 9B, an Edge-to-Edge device, such as a clip, is assumed, so that a rectangular range is set each of the anterior leaflet and the posterior leaflet. The rectangular range is used by an estimation process that will be described later as an area in which the anterior leaflet and the posterior leaflet are connected by the treatment device.

Then, the identification function 25e identifies the attention grid on the basis of the range identified in FIG. 9B. That is, the identification function 25e is able to identify the attention grid on the basis of the display condition (the display angle and the position of the slice direction) related to the cross-sectional position of the image I1 and the size of the treatment device.

For example, the identification function 25e identifies all of the grid points that are located within the identified range as a candidate for the attention grid. In the case illustrated in FIG. 9B, for example, the grid of the anterior leaflet indicated by the identifiers (X, Y) of (4, 3), (3, 3), (2, 3), (4, 4), (3, 4), and (2, 4), and the grid of the posterior leaflet indicated by the identifiers (X, Y) of (4, 13), (3, 13), (2, 13), (4, 14), and (3, 14) are identified as the candidates for the attention grids. These candidates for the attention grids depend on the image display condition of the attention images. For example, in the case where the image display area in which the attention image is displayed has been changed among the areas 303b to 303d or in the case where an operation of a slice feed (browse) has been performed, the candidates for the attention grids are sequentially updated in accordance with the image display condition of the changed attention image. Then, the identification function 25e identifies, as the attention grids, the candidates for the attention grids at the time of a “Yes” button indicated in an area 400c being pressed by the user.

The grid point ID of the identified attention grid is displayed in each of the fields of the “Anterior” and the “Posterior” indicated in the area 400c. Alternatively, the grid point ID of the candidate for the attention grid may be displayed in each of the fields of the “Anterior” and the “Posterior”. In this case, the display of each of the fields of the “Anterior” and the “Posterior” is sequentially updated every time the image display condition of the attention image is changed.

Alternatively, the identification function 25e may identify the attention grid by receiving an input of the grid point ID with respect to each of the fields of the “Anterior” and the “Posterior” from the user. For example, the display control function 25d displays the grid point ID of the grid point corresponding to the position of the mouse cursor when the mouse cursor is overlaid on the mesh that is displayed in the area 303a illustrated in FIG. 8A. The user is able to input the grid point ID to each of the fields of the “Anterior” and the “Posterior” while referring to the displayed grid point ID. Furthermore, the display control function 25d may be configured such that the display of the grid point ID in accordance with the position of the mouse cursor is allowed only when the “Simulation” is selected in the area 400a, and the display of the grid point ID is not allowed when the “Measurement” is selected.

Furthermore, an area 400d illustrated in FIG. 8A receives a result save name that is to be set. As the initial value of the area 400d, “Sim_+Case ID_+#” may be displayed. Here, in the part of “Sim_”, for example, a prefix number that is used to identify the type of data is input. Furthermore, in the part of “Case ID”, for example, the ID corresponding to a case in which a simulation has been performed is input from among the IDs that are preset for each case. The symbol “#” is incremented in accordance with the number of results in each of which a simulation is performed on the subject case. Moreover, for example, in the case where the format of the setting in the areas 400b to 400d illustrated in FIG. 8A is not correct, such as a case in which a data entry other than a value is input to the fields of the “Anterior” and the “Posterior”, or the field is blank, it may be possible to display a message for prompting the user to perform setting again.

In FIG. 9B, the case has been described as the example in which all of the grid points that are located within the range that has been identified on the basis of the size of the image I1 and the treatment device are identified as the attention grid, but the embodiment is not limited to this. For example, the identification function 25e may identify, as the attention grid, the grid points in each of the columns that are closest to the identified range.

Furthermore, in FIG. 9B, the example in which the size of the range is determined on the basis of the size of the treatment device has been described, but the embodiment is not limited to this. For example, in the case where the image I1 corresponding to the attention image is a slab MIP image having the width that is based on the width specified by the user, the identification function 25e may identify the range in which the width of the slab MIP image is used instead of the width “B” illustrated in FIG. 9B, and identify the attention grid on the basis of the identified range.

In addition, a method of setting the attention grid and the type of the treatment device that can be set is not limited to the example described above. For example, in the case where an artificial valve device in a valve replacement surgery is used, the identification function 25e selects a plurality of two-dimensional images each having a different display angle as the attention images. For example, the identification function 25e selects an image 12 and an image 13 illustrated in FIG. 9C as the attention images. Then, the identification function 25e identifies a circle that has a radius of “z” that is determined by the size of the artificial valve device and that is centered at the position of the intersection point between the image 12 and the image 13. The radius “z” may be set on the basis of an enlargement percentage of the attention image. Furthermore, as described above, the size of the artificial valve device is able to be set by the user.

Then, the identification function 25e identifies the attention grid on the basis of the identified circle with the radius of “z”. For example, the identification function 25e identifies, as indicated by circular marks illustrated in FIG. 9D, the grid point in each of the columns that are closest to the position of the circumference of the identified circle as the attention grid. Specifically, in FIG. 9D, the grid indicated by the identifiers (X, Y) of (2, 0), (2, 1), (2, 2), (1, 3), (1, 4), (1, 5), (1, 6), (1, 7), (1, 8), (1, 9), (2, 10), (2, 11), (2, 12), (2, 13), (2, 14), (2, 15), (2, 16), (2, 17), (2, 18), and (2, 19) is identified as the attention grid. As another example, the identification function 25e may identify, as the attention grid, the grid points that are included in the range that has a constant width from the center of the identified circle around the circumference.

In the case where a plurality of attention images are set, the display control function 25d may perform a display in accordance with the subject setting. For example, in FIG. 8A, the case has been described as the example in which the single piece of the image I1 that has been selected as the attention image is highlighted, but the display control function 25d may also highlight a plurality of images that are selected as the attention image. At this time, the display control function 25d may change the display condition for each image such that the order of the selected images can be identified (for example, the color of the frame of each of the selected images are changed, etc.).

Various modifications are possible for the method of identifying the attention grid. For example, the identification function 25e is also able to identify the attention grid on the basis of the center position of the attention image. As one example, the identification function 25e is able to identify the grid point corresponding to the center position of the attention image, and identify the grid points that are included in a certain range from the identified grid point as the attention grid.

Furthermore, the identification function 25e is able to identify the attention grid on the basis of the center position of the attention image and the enlargement percentage. As one example, the identification function 25e is able to identify the grid point corresponding to the center position of the attention image, and identify, from the identified grid point as the attention grid, the grid points that are included in the range having the size that is in accordance with the enlargement percentage of the attention image. For example, the identification function 25e sets a smaller range as the enlargement percentage is larger, and identifies the grid points that are included in the range as the attention grid.

Furthermore, the identification function 25e is able to identify the attention grid on the basis of the display condition related to the display color of the WW, the WL and the like. For example, the WW and the WL by which various kinds of organs are easily visible are generally determined for each organ, so that the identification function 25e sets in advance the correspondence relationship between the values of the WW and the WL and the various kinds of organs. For example, the identification function 25e records the values of the WW and the WL that are manually set by the user at the time of observation of the mitral valve, associates the average value of the recorded values with the organ “mitral valve”, and records the associated data. Accordingly, the identification function 25e is able to identify the organ that is targeted for the observation on the basis of the values of the WW and the WL that are set as the display condition, identifies the position of the organ targeted for the observation from the medical image data, and identify the attention grid on the basis of the position of the identified organ.

The identified attention grid may also be highlighted in the image display area, such as the areas 303a to 303d. For example, the display control function 25d highlights the attention grid by changing the color of the attention grid in the mesh or changing the color of the position corresponding to the attention grid in the VR image, the MPR image, and the like.

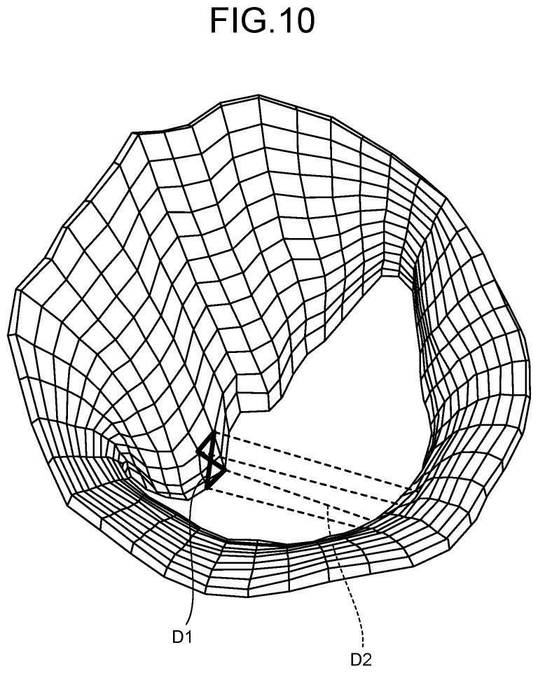

Furthermore, as illustrated in FIG. 10, the display control function 25d may also display a mark corresponding to the estimation process that is performed at Step S7, which will be described later, on the basis of the identified attention grid. In FIG. 10, a position D1 and a plurality of straight lines D2 are illustrated with respect to the three-dimensional mesh. The position D1 indicates the position (clip position) in which the clip is placed by percutaneous mitral valve clip surgery. In addition, the plurality of straight lines D2 indicate the relationship between the grid points that are connected by the clip. Here, the position D1 and the straight lines D2 are determined on the basis of the identified attention grid. For example, the position D1 is an area of a polygon obtained by connecting the attention grid. Furthermore, each of the straight lines D2 are obtained by connecting the grids of the anterior leaflet and the grids of the posterior leaflet included in the attention grid. For example, as a result of a selection of the icon (the icon illustrated in FIG. 8B and FIG. 8C) that is located adjacent to the “VR View” indicated in the area 400c illustrated in FIG. 8A, a display/non-display of the plurality of straight lines D2 is switched.

Furthermore, the display control function 25d may also display a simulated device (for example, a 3D model indicating the shape of the clip, etc.) with respect to the three-dimensional mesh on the basis of the position of the identified attention grid.

Furthermore, the display control function 25d may also highlight the identified attention grid on the MPR image that is displayed in, for example, the areas 303b to 303d. For example, as a result of a selection of the icon (the icon illustrated in FIG. 8B and FIG. 8C) that is located adjacent to the “MPR View” indicated in the area 400c illustrated in FIG. 8A, the display control function 25d determines whether the attention grid is highlighted on the MPR image, and switches the state in accordance with the determination result.

For example, in the case where the MPR image including the position of the identified attention grid is displayed in each of the areas 303b to 303d and the icon illustrated in the FIG. 8C is selected, the display control function 25d highlights the mark that indicates the attention grid and the connection lines of the attention grid on the MPR image. For example, the display control function 25d highlights the attention grid by displaying only the attention grid by omitting the display of the grid points other than those of the attention grid. Alternatively, the display control function 25d highlights the attention grid by displaying the attention grid by a mark having the color and the size that are different from those of the grid points other than the grid points of the attention grid. Alternatively, the display control function 25d may also display, on the MPR image, the mark that indicates the intersection point between the connection line of the attention grid and the MPR image. Furthermore, even when the icon illustrated in FIG. 8C has been selected, if the MPR image including the position of the attention grid is not displayed in the areas 303b to 303d, the display control function 25d does not need to highlight the attention grid on the MPR image.

Then, the identification function 25e determines whether or not the process of identifying the attention grid is to be completed (Step S6). For example, the identification function 25e receives an operation from the user with respect to the GUI indicating whether or not the process of identifying the attention grid is to be completed. Here, if the process of identifying the attention grid is not completed (No at Step S6), the process proceeds to Step S3 again, and the processes at Step S3 to S6 are repeated. In other words, the display condition of the medical image data is changed, the medical image data is displayed under the changed display condition, and the attention grid is again identified on the basis of the display condition of the displayed medical image data. Furthermore, the determination performed at Step S6 has been described as the determination whether or not the process of identifying the attention grid is to be completed, but the determination may be replaced with the determination whether or not the process at Step S7 is started.

Then, the processing function 25f performs a physical simulation by using the attention grid identified by the identification function 25e as the calculation condition (Step S7). For example, the processing function 25f performs the physical simulation on the basis of the grid point cloud data that has been identified at Step S2, the attention grid that has been identified at Step S5, and various kinds of parameters (including the boundary condition) that are used for the physical simulation that is defined in advance.

The physical simulation performed by the processing function 25f is started when, as a trigger, for example, an icon 400e illustrated in FIG. 8A has been selected. Furthermore, in the case where the physical simulation is not normally ended and an error has been responded from the simulation engine included in the processing function 25f, the display control function 25d may also display the message in accordance with the error. For example, the simulation engine outputs an error code, and the display control function 25d generates and displays a message on the basis of the error code. Furthermore, for example, the simulation engine outputs a message in accordance with the error, and the display control function 25d displays the output message.

In the following, a case will be described in which grid point cloud data related to the mitral valve has been acquired from the medical image data on the mitral valve as the target organ acquired before treatment. In this case, the processing function 25f estimates the shape of the mitral valve obtained after the treatment in which the Edge-to-Edge device with the type that has been specified by the user is placed at the position corresponding to, for example, the attention grid. A known method may be used for this estimation. Examples of the known method includes, for example, a finite element method, a finite difference method, an immersed boundary method, and the like. More specifically, parameters based on the treatment device are set to the attention grid that has been identified at Step S5. For example, the processing function 25f sets a virtual spring with respect to the attention grid, and estimates a change in the shape while changing the spring constant of the spring. Then, the change in the spring constant is stopped at the time at which the anterior leaflet and the posterior leaflet have been connected. The shape at the time of a change in the spring constant is able to be estimated by using, in addition to the attention grid, a mathematical model or a physical model that is set to the other grid points.

The process of the processing function 25f described above is one example, and any method may be used as long as a movement of an object and information related to a fluid can be estimated. For example, it may be possible to estimate a post-treatment shape of the target area from a shape model that has been built by learning data that is used for learning and that is prepared in advance by using a machine learning technology, such as deep learning. Any method may be used for the estimation process, but there is a need to use a method in which a parameter that is different from the other grids, or a different a mathematical model or a different physical model can be used for the attention grid that has been identified at Step S5. Any parameter may be used for the parameter that is used for the estimation, and, furthermore, in addition to the parameter based on the treatment device, it may be possible to set a parameter based on an anatomical structure, such as the position of a chorda tendinea, the number of chordae tendineae, and tension, or a fluid parameter, such as a blood flow distribution. The various kinds of parameters may be set in advance, or the method described in Patent Literature 4 may be used to identify the attention grid.

In the above, the example in which a post-treatment shape is estimated as a physical simulation has been described, but, in addition to the shape, a state or a force of the fluid at the time of post-treatment may be estimated. The fluid is, for example, a blood flow. Examples of the state of the blood flow include a forward blood flow rate, a backward blood flow rate, a blood flow field, and the like. Furthermore, examples of the force include a pressure distribution caused by a blood flow related to the valve leaflet, tension of a chorda tendinea, and the like.

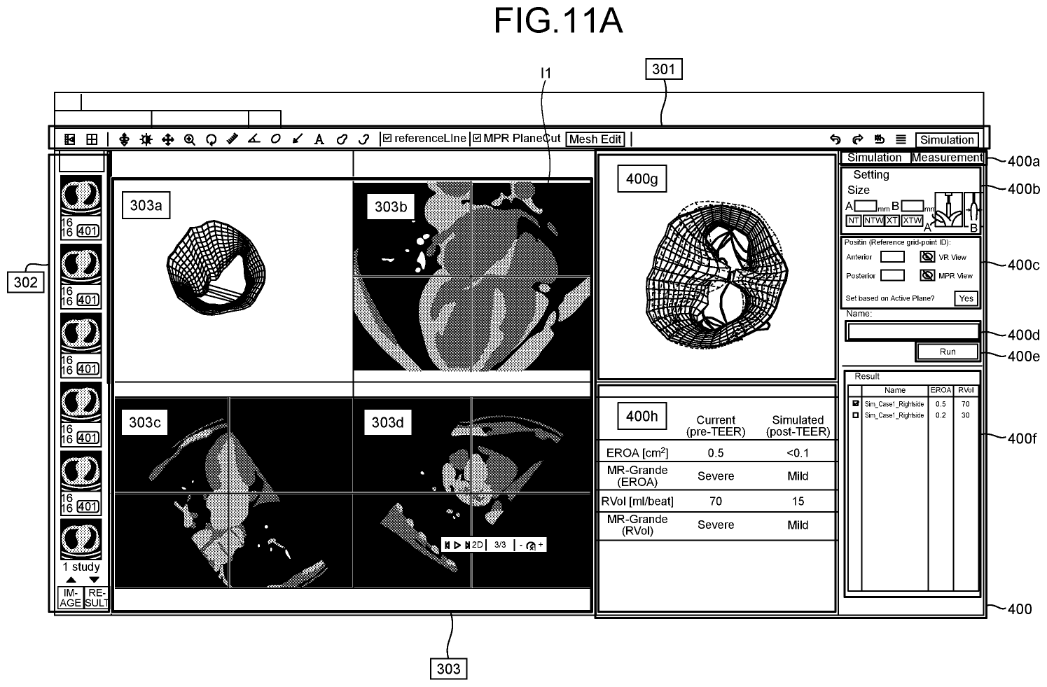

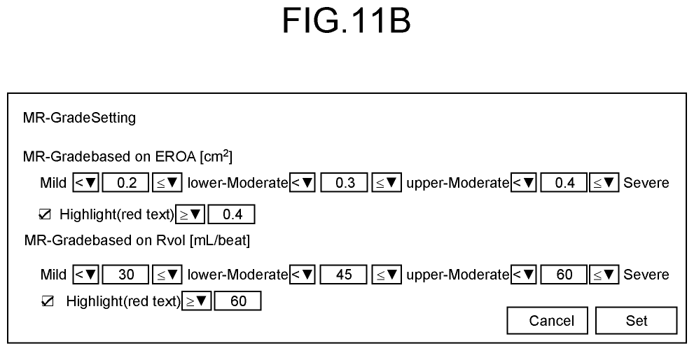

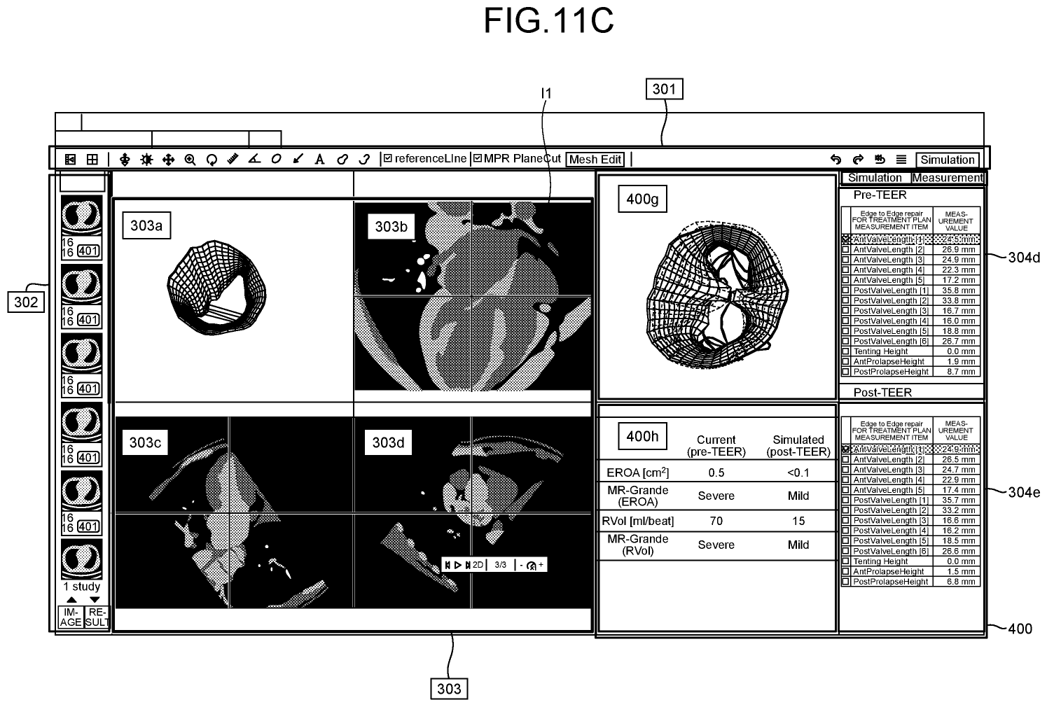

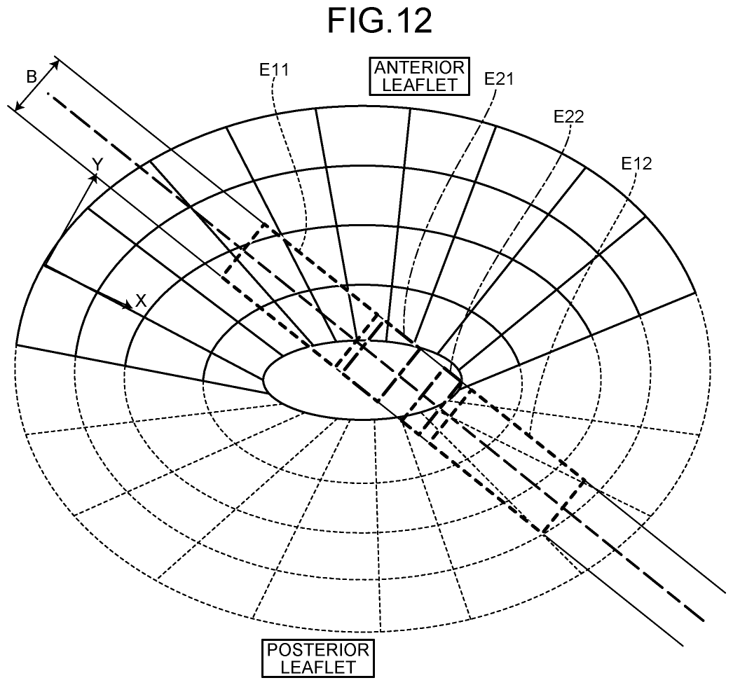

FIG. 11A illustrates one example of a display of the results of the physical simulation. In an area 400f, a list of the simulation results (Result) is displayed. That is, the results of the simulation that has been calculated once is stored. It is possible to display, in areas 400g and 400h, the result indicated by a check mark in the checkbox from among the displayed simulation results included in the list. It may be possible to display the simulation condition, such as a “Clip Size”, a “position”, and a “Name”, bringing about the simulation results corresponding to the areas 400b and 400c may be displayed in the area 400b, the area 400c, or the area 400d. The list in the area 400f may be sorted in accordance with the item that has been selected from among the items, such as “Name”, “EROA”, “RVol”, on the basis of an ascending order or a descending order of values, or the like. Furthermore, in the area 400f illustrated in FIG. 11A, two simulation results of “Sim_Case1_Rightside” and “Sim_Case1_Rightside” are displayed. The name of each of the simulation results corresponds to the name (result save name) that is input to the area 400d when the icon 400e has been selected. The items of “EROA” and “RVol” will be described later.

The simulation results that are included in the list indicated in the area 400f may be configured to be deletable as appropriate. For example, the structure may be configured to display the context menu by a right click and an arbitrary result can be deleted from the context menu. Furthermore, it may be possible to provide a deletion button (not illustrated), and delete, when the user selects the result to be deleted and then selects the deletion button, the selected result. Moreover, when the physical simulation is ended without any problems, the user adds the result to the list in the area 400f and checks the corresponding checkbox to display the results in the areas 400g and 400h.

Furthermore, in the area 303a illustrated in FIG. 11A, the mesh that indicates the shape of the mitral valve at the time of pre-treatment is displayed. Regarding the mesh displayed in the area 303a, similarly to FIG. 10, it may also be possible to display the relationship between the clip position and the grid points that are connected by the clip.

Furthermore, in the area 400g illustrated in FIG. 11A, a mesh that indicates the shape of the mitral valve obtained at the time of post-treatment estimated by the physical simulation is displayed. Regarding the mesh displayed in the area 400g, it is possible to use the function performed by using various kinds of icons that are displayed in the area 301. For example, regarding the mesh displayed in the area 400g, the user is able to change the display color by the function of the icon 301d, perform a parallel shift by the function of the icon 301e, change the enlargement percentage by the function of the icon 301f, change the display angle by the function of the icon 301g, and the like. Moreover, in FIG. 11A, the mesh is displayed in each of the area 303a and the area 400g, but the mesh may also be replaced with a VR image or the like in accordance with an instruction received from the user.

Furthermore, in the table displayed in the area 400h illustrated in FIG. 11A, values based on the physical simulation result and “MR-Grade” based on the values are displayed. The “MR-Grade” indicates the degree of mitral valve insufficiency. For example, the “MR-Grade” is divided into four grades of “Mild”, “lower-Moderate”, “upper-Moderate”, and “Severe” in accordance with the degree of the mitral valve insufficiency.

The “EROA (effective regurgitant orifice area)” displayed in the area 400h is a value that is measured from the shape of the mesh displayed in, for example, each of the area 303a and the area 400g. For example, in FIG. 11A, pre-treatment “Current (pre-TEER)” is a value that is calculated on the basis of the mesh (the grid point cloud data that has been acquired at Step S2) that is being displayed in the area 303a, and an example in which “EROA=0.5” is displayed as the calculation result is illustrated. In contrast, simulated post-treatment “Simulated (post-TEER)” is a value that is estimated by the simulation performed at Step S7, and an example in which “EROA<0.1” is displayed as the estimation result is illustrated. Accordingly, the “MR-Grade (EROA)” that is the “MR-Grade” based on the “EROA” is improved to “Mild” at the time of the post-treatment as compared to “Severe” at the time of the pre-treatment. In other words, from a viewpoint of the “EROA”, it is estimated that a sufficient treatment effect can be obtained by placing the clip as planned at, for example, the position D1 illustrated in FIG. 10.

Furthermore, “RVol (backward blood flow rate)” is calculated from the physical simulation, such as fluid analysis, performed by using the shape of the mesh displayed in, for example, each of the area 303a and the area 400g. For example, in FIG. 11A, pre-treatment “Current (pre-TEER)” is a value that is estimated by the physical simulation performed by using the shape of the mesh (the grid point cloud data that has been acquired at Step S2) that is displayed in the area 303a, and an example in which “RVol=70” is displayed as the calculation result is illustrated. In contrast, the simulated post-treatment “Simulated (post-TEER)” is a value that is estimated by the physical simulation performed by using the shape of the mesh that has been estimated by the simulation performed at Step S7, and an example in which “RVol<15” is displayed as the estimation result is illustrated. Accordingly, the “MR-Grade (RVol)” that is the “MR-Grade” based on the “RVol” has been improved to “Mild” at the time of the post-treatment as compared to “Severe” at the time of the pre-treatment. In other words, from a viewpoint of the “RVol”, it is estimated that a sufficient treatment effect can be obtained by placing the clip as planned at, for example, the position D1 illustrated in FIG. 10.

A criterion (threshold) for determining the “MR-Grade” with respect to the values, such as the “EROA” and the “RVol”, may be configured to be able to be set by using a UI illustrated in, for example, FIG. 11B. For example, in FIG. 11B, the configuration has been set such that the range of a “EROA<0.2” indicates “Mild”, a range of “0.2≤EROA<0.3” indicates “lower-Moderate”, a range of “0.3≤EROA<0.4” indicates “upper-Moderate”, and a range of “0.4≤EROA” indicates “Severe”. Furthermore, the configuration has been set such that the range of “RVol<30” indicates “Mild”, a range of “30≤RVol<45” indicates “lower-Moderate”, a range of “45≤RVol<60” indicates “upper-Moderate”, and a range of “60≤RVol” indicates “Severe”. It may also be possible to set the values illustrated in FIG. 11B as the initial setting and receive a change of the threshold from the user. For example, after an operation of left clicking the threshold displayed in FIG. 11B has been performed, by receiving an input of a value via a keyboard, it may also be possible to replace the threshold with the value that has been input via the keyboard. Furthermore, for example, after an operation of left clicking the threshold, by receiving an operation of rotating the wheel of the mouse, it may also be possible to increase or decrease the threshold in accordance with an amount of rotation of the wheel and the rotational direction. Moreover, for example, an icon (not illustrated) is displayed in the vicinity of the threshold, and the value may also be increased or decreased in accordance with the operation performed on the icon. In addition, the changed threshold may also be stored and used at the next physical simulation and the subsequent physical simulations.

Furthermore, “<”, and “>” are inequality signs. For example, “x1<x2” indicates that “x1” is smaller than “x2” and also indicates that “x1” and “x2” are not equal. Furthermore, “x1>x2” indicates that “x1” is larger than “x2” and also indicates that “x1” and “x2” are not equal. Furthermore, “≤”, and “≥” are each inequality sign with equal sign. For example, “x1≤x2” indicates that “x1” is smaller than “x2”, or indicates that “x1” and “x2” are equal. Furthermore, “x1≥x2” indicates that “x1” is larger than “x2”, or indicates that “x1” and “x2” are equal.

When the checkbox of “Highlight” illustrated in FIG. 11B has been checked, the value that satisfies the set condition the characters of the “MR-Grade” is highlighted by changing, for example, the character color, or the like. In FIG. 11A and FIG. 11B, the range of “0.4≤EROA” and the range of “60≤RVol” (i.e., the range corresponding to the “Severe”) are set as the targets for being highlighted.

Furthermore, when a change in signs, such as the inequality sign or the inequality sign with equal sign, is received, it may also be possible to perform control such that only a combination of “≤” and “<” that are disposed on both sides of each of the threshold is selectable. For example, in FIG. 11B, at the setting of the “MR-Grade” based on EROA, the threshold is set to “0.4” and the relationship of “upper-Moderate<0.4≤Severe” is set. When the sign is changed at this time, no particular problem occurs as long as the relationship is changed to “upper-Moderate≤0.4<Severe”, but, if the relationship is changed to “upper-Moderate≤0.4≤Severe” or changed to “upper-Moderate<0.4<Severe”, an overlap or an omission of the value range for each category, such as the “upper-Moderate” and the “Severe”, occurs. Furthermore, it may also be configured such that the setting of the orientation of the sign is not able to be changed. For example, in the case where the relationship of “upper-Moderate<0.4≤Severe” has been set, if a change, such as “upper-Moderate<0.4>Severe” or “upper-Moderate<0.4≥Severe”, is performed, an overlap occurs in the value range for each category. Therefore, it may be possible to construct the configuration such that this sort of setting is not allowed, or, it may be possible to construct the configuration such that, if this sort of setting has been performed, a display that indicates to urge the user to modify the setting may be displayed. For example, if one side is “<”, the other side may be automatically set to “≤”, whereas, if one side is “≤”, the other side may be automatically set to “<”. Furthermore, it may be possible to construct the configuration such that an arbitrary value can be input but a value that does not maintain the relationship of “<” or the relationship of “≤” is not able to be set. In other words, it may also be possible to perform control so as not to conflict the magnitude relationship between the values defined by the inequality sign or the inequality sign with equal sign. For example, in FIG. 11B, the threshold between the “Mild” and the “lower-Moderate” based on “EROA” is set to “0.2”, and the threshold between the “lower-Moderate” and the “upper-Moderate” based on “EROA” is set to “0.3”. Here, it may also be possible to perform control such that, when the threshold of “0.2” between the “Mild” and the “lower-Moderate” is changed, a changeable value range is set to “<0.3”. Alternatively, it may also be possible to perform control such that, when the value of “0.3≤” is input, a message that urges to change the value may be displayed.

When, for example, the “Measurement” displayed in the area 400a has been selected, the display control function 25d may replace a part of the area 400 or the entire area 400 with the area 304 in which the measurement results are displayed. A display example is illustrated in FIG. 11C. In FIG. 11C, the area 304 is displayed by leaving the area 400g and the area 400h included in the area 400. Furthermore, in FIG. 11C, in the area 304, an area 304d for displaying the measurement result obtained before treatment and an area 304e for displaying the simulated measurement result obtained after the treatment are displayed.

For example, the display control function 25d displays, in the area 304d, various kinds of measurement values that are based on the shape of the mesh at the time of pre-treatment displayed in the area 303a as a list by associating the measurement values with the various kinds of measurement item names. Furthermore, the display control function 25d displays, in the area 304e, various kinds of measurement values that are based on the shape of the simulated mesh obtained at the time of post-treatment in the area 400g as a list by associating the measurement values with the various kinds of measurement item names. Here, if the display condition (for example, a cardiac phase, etc.) has been changed, the various kinds of measurement values displayed in the area 304d and the area 304e are updated in accordance with the changed display condition.

However, there may be a case in which, regarding the measurement values based on the simulation results that are displayed in the area 304e, data corresponding to all of the phases are not generated. For example, if the controller 305 related to a cine feed is operated and an instruction to display the phase whose data has not been generated is input, it may also be possible to display a message indicating that “no simulation result is present”. Furthermore, regarding the measurement values based on the simulation result, a display corresponding to the “VR view” and the “MPR View” illustrated in FIG. 8A is not needed.

The measurement value to be displayed in the area 304e may be displayed as soon as the simulation has been completed, or may be measured when an instruction is received from the user after the completion of the simulation or may be measured after an elapse of predetermined time. For example, it may also be possible to display, with priority, the simulation results indicated in the area 400 at the time of completion of the simulation, and start a measurement after the user has checked the simulation results.

As described above, the image data acquisition function 25b acquires the medical image data including the target organ. Furthermore, the grid point cloud data acquisition function 25c acquires the grid point cloud data that is related to the target organ and that is associated with the medical image data. Furthermore, the display control function 25d displays the medical image data. Furthermore, the identification function 25e identifies an attention grid included in the grid point cloud data on the basis of the display condition of the medical image data. Consequently, the user is able to easily identify the attention grid that is used to perform the simulation that will be described later.

As another method of identifying the attention grid, it is conceivable to display the grid point cloud data (for example, a mesh) related to the target organ and receive an operation of specifying the attention grid from the user. However, this sort of operation is complicated for the user, and, furthermore, the correspondence relationship between each of the grid points and the structure of the actual organ is not displayed, so that it is difficult to perform an intuitive operation. In contrast, according to the above described process performed by the medical information processing apparatus 20, the user is able to identify the attention grid by adjusting the display condition of the medical image data while referring to the medical image data. In other words, in the above described process performed by the medical information processing apparatus 20, the user is able to easily identify the attention grid by performing a simple and intuitive operation.

In the embodiment described above, a case has been described as an example in which the target organ is a valve, but the type of the target organ is not particularly limited. For example, it may be possible to perform the process at each of the steps illustrated in FIG. 2 by using a blood vessel, a lung, a liver, or the like of the subject as the target organ. As one example, by performing the process at each of the steps illustrated in FIG. 2, it may be possible to identify the attention grid corresponding to the placement position of a catheter in catheter treatment of a coronary artery, and performing the physical simulation for estimating the state of the coronary artery after the catheter treatment. Furthermore, as one example, it is possible to identify the attention grid corresponding to an excision area of the lung or the liver by the process performed at each of the steps illustrated in FIG. 2, and perform a physical simulation for estimating the state of the lung or the liver after the excision process.

Furthermore, in FIG. 9B, a case has been described as an example in which, on the basis of the display condition that is related to the cross-sectional position of the displayed medical image data and the size of the treatment device, after the range has been determined with respect to each of the anterior leaflet and the posterior leaflet, the grid point cloud that is located within each of the determined ranges is identified as the attention grid. However, the embodiment is not limited to this.

For example, the identification function 25e may determine a plurality of ranges on the basis of the display condition, and set, on the basis of each of the plurality of ranges, a plurality of attention grids that are used to set a different or the same condition (boundary condition) at Step S7. For example, as illustrated in FIG. 12, in the case where, in the range that is set on the basis of the display condition and the size of the treatment device, grid points are included in both of a range E21 and a range E22 that are located on the inner side (valve tip side) than the positions of the range E11 and the range E12 corresponding to the clip positions, the identification function 25e may identify the subject grid points as a second attention grid. Furthermore, in FIG. 12, the grid point indicating that the identifier (X, Y) corresponds to (4, 5) is included in the range E21, the grid point indicating that the identifier (X, Y) corresponds to (4, 12) is included in the range E22, and these grid points are identified as the second attention grid.

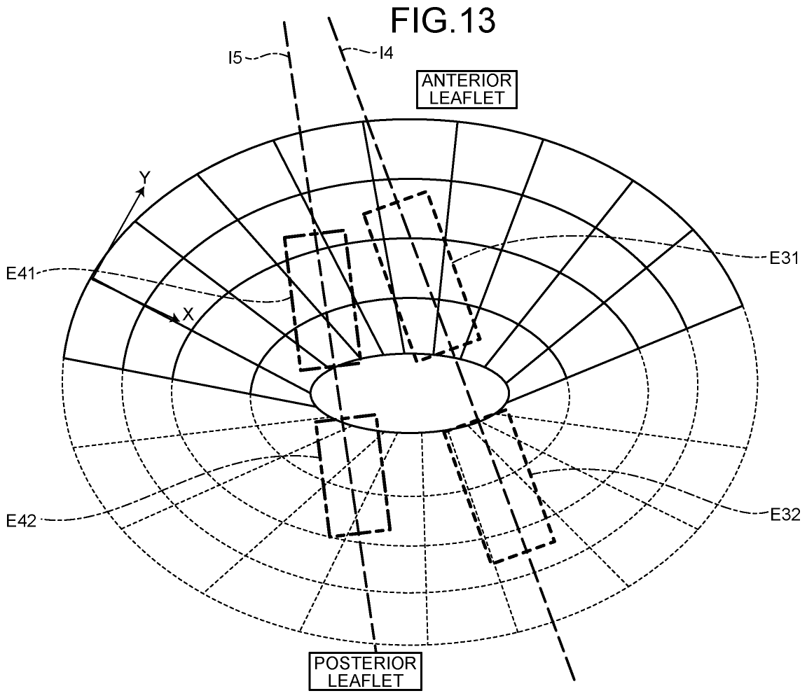

In FIG. 12, the method of identifying the plurality of different attention grids by using the same display condition has been described, but it may be possible to identify a plurality of different attention grids by using a plurality of different display conditions and treatment device conditions. For example, in FIG. 13, a range E31 and a range E32 are set on the basis of the display condition of an image 14, and the grid points located within these ranges are identified as a first attention grid. Furthermore, in FIG. 13, a range E41 and a range E42 are set on the basis of the display condition of an image 15, and the grid points located within these ranges are identified as a second attention grid. The process performed in FIG. 13 may be used in the case where, for example, two clips are placed. In other words, it may be possible to perform a physical simulation for estimating a state of the valve after having performed treatment in which a first clip is placed at the position of the first attention grid and a second clip is placed at the position of the second attention grid. Furthermore, in the case where the grid points are overlapped in the first attention grid and the second attention grid, it may also be possible to perform control such that an error message is output. The overlap of the grid points corresponds to an interference between the two clips.

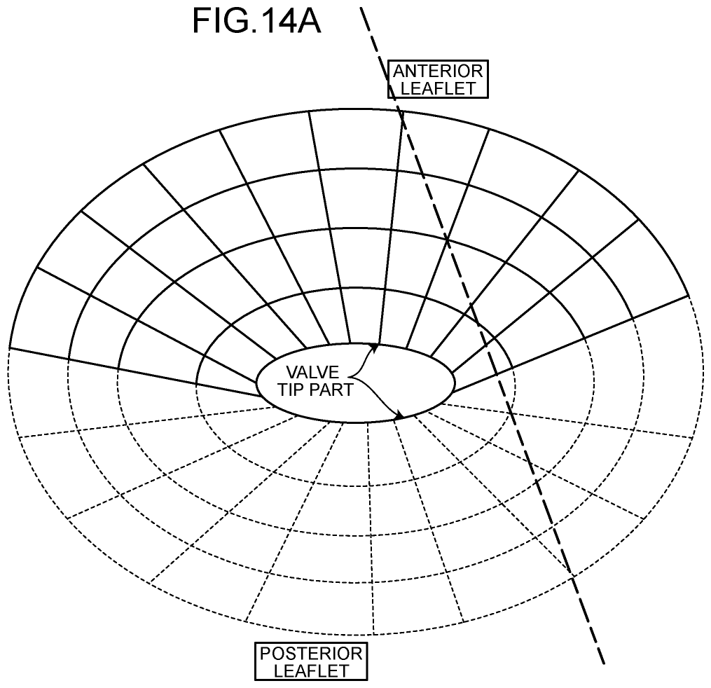

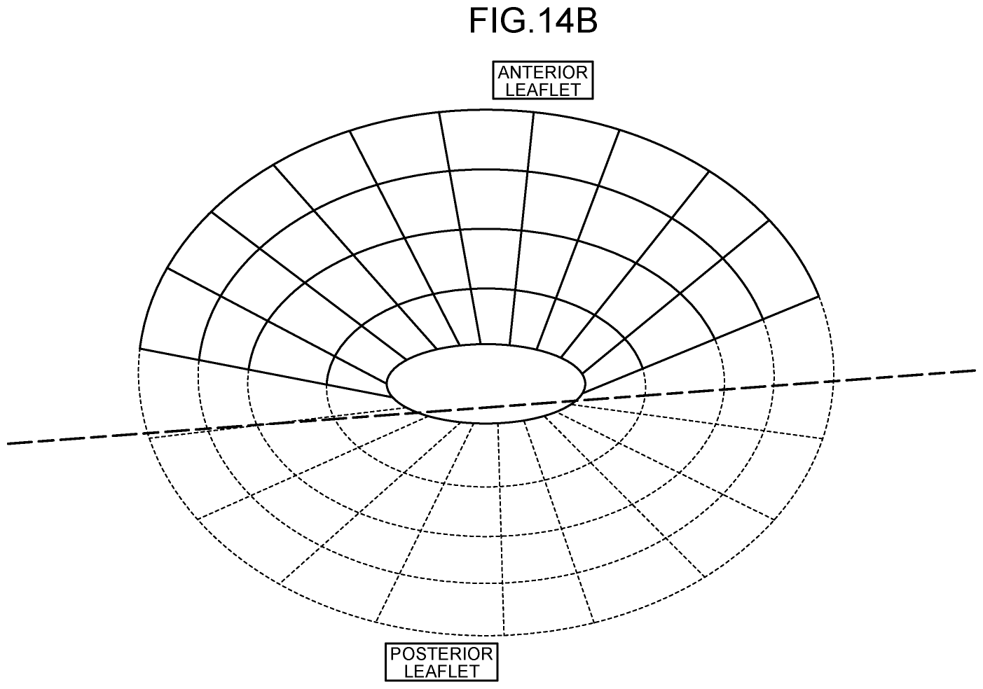

Furthermore, at Step S5 illustrated in FIG. 2, in the case where the display condition that has been set at Step S3 is a condition that is unsuitable for identifying the attention grid, the identification function 25e may display a message that urges the user to perform a modification. For example, as illustrated in FIG. 14A, in the case where the cross-sectional position of the attention image does not pass through the valve orifice (an area surrounded by the valve tip part), or, as illustrated in FIG. 14B, in the case where the cross-sectional position of the attention image passes through the valve orifice but only passes through one of the areas of the anterior leaflet and the posterior leaflet, it is not suitable for the position in which a click is placed, and thus, the identification function 25e may display a message that urges the user to perform a modification. It is possible to set such an unsuitable condition in advance for, for example, each type of the treatment device or each organ. Furthermore, it is possible to determine whether or not the cross-sectional position of the attention image passes through the valve orifice by determining whether or not the cross-sectional position passes through the valve tip part.

In FIG. 1, it has been described that the various kinds of functions, such as the control function 25a, the image data acquisition function 25b, the grid point cloud data acquisition function 25c, the display control function 25d, the identification function 25e, and the processing function 25f are implemented by the processing circuitry 25 included in the medical information processing apparatus 20, but these function may also be distributed to a plurality of devices as appropriate. For example, the processing function 25f may also be implemented by processing circuitry included in a second medical information processing apparatus that is different from the medical information processing apparatus 20. In this case, the medical information processing apparatus 20 identifies the attention grid, and notifies the second medical information processing apparatus of the identified attention grid. In the second medical information processing apparatus, a physical simulation is performed by using the notified attention grid as the calculation condition. Furthermore, each of the functions in the processing circuitry 25 may also be implemented by a processing circuit that is provided with the console device in, for example, the medical image diagnostic apparatus 10. In other words, the medical image diagnostic apparatus 10 and the medical information processing apparatus 20 may also be integrated with each other.

Furthermore, in the embodiment described above, it has been described about, as an example of the medical image data, a plurality of pieces of time-series CT image data (four-dimensional image), but the embodiment is not limited to this. For example, it is possible to perform the process of each of the steps illustrated in FIG. 2 on the basis of the pieces of CT image data (three-dimensional image) that are collected about a single phase. Moreover, in a case of a three-dimensional image, for example, some function, such as a cine feed, performed by using the controller 305 is omitted.

Furthermore, it may also be possible to acquire a two-dimensional image as the medical image data and perform the process at each of the steps illustrated in FIG. 2. In this case, the grid point cloud data acquired at Step S2 corresponds to the data that includes each of the position coordinates of the plurality of grid points on a certain plane. Furthermore, at Step S7, a two-dimensional simulation on a certain plane is performed.

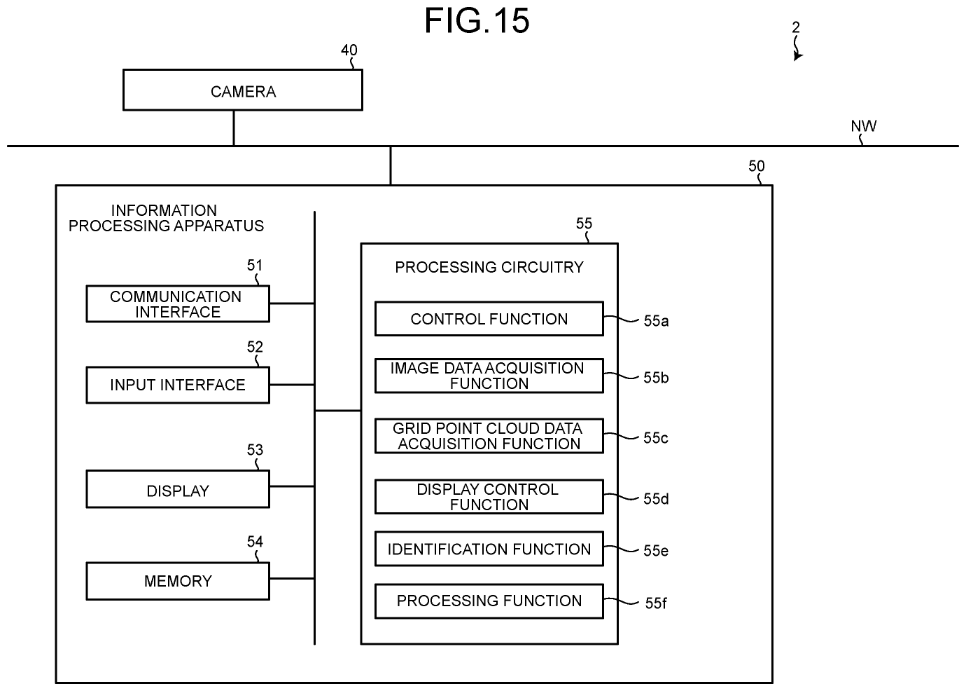

Furthermore, it has been described that the medical image data is acquired at Step S1, but it is also possible to similarly perform the processes at Steps S2 to S7 in the case where image data other than the medical image data is acquired. In the following, this point will be described with reference to FIG. 15. FIG. 15 is a block diagram illustrating one example of a configuration of an information processing system 2 according to the embodiment.

As illustrated in FIG. 2, the information processing system 2 includes a camera 40 and an information processing apparatus 50. The camera 40 is, for example, and optical camera. In this case, the camera 40 is able to capture an image of the image data on the surface of the body of the subject, and transmits the obtained data to the information processing apparatus 50. The image data captured by the camera 40 may be captured by aiming at treatment or the like of a disease held by the subject, or may be captured by targeting the subject who does not a particular disease from a viewpoint of, for example, sports science.

For example, as illustrated in FIG. 15, the information processing apparatus 50 includes a communication interface 51, an input interface 52, a display 53, a memory 54, and processing circuitry 55. It is possible to configure the communication interface 51, the input interface 52, the display 53, and the memory 54 in a similar manner as for the communication interface 21, the input interface 22, the display 23, and the memory 24 illustrated in FIG. 1. Furthermore, the processing circuitry 55 performs a control function 55a, an image data acquisition function 55b, a grid point cloud data acquisition function 55c, a display control function 55d, an identification function 55e, and a processing function 55f.

The control function 55a is the same function as the control function 25a. The image data acquisition function 55b is the same function as the image data acquisition function 25b, and is also one example of an image data acquisition unit. The image data acquisition function 25b acquires, via the network NW, the image data including the target object captured by the camera 40. For example, the camera 40 captures image data of a specific muscle in the subject, and a region of an upper arm, a lower limb, or the like as the target object. The image data acquisition function 25b may also directly acquire the image data from the camera 40, or may also acquire the image data that is stored in the storage apparatus, such as the image storage apparatus 30.

The grid point cloud data acquisition function 55c is the same function as that of the grid point cloud data acquisition function 25c, and is also one example of a grid point cloud data acquisition unit. For example, the grid point cloud data acquisition function 55c acquires, on the basis of the image data on the surface of the body of the subject, the grid point cloud data in which the plurality of grid points corresponding to the surface of the body are arranged in a curved shape. The display control function 55d is the same function as the display control function 25d, and is also one example of a display control unit. The identification function 55e is the same function as the identification function 25e, and is also one example of an identification unit. The processing function 55f is the same function as the processing function 25f, and is also one example of a processing unit.

The term “processor” used in the above description indicates, for example, a circuit, such as a CPU, a graphics processing unit (GPU), an application specific integrated circuit (ASIC), a programmable logic device (for example, a simple programmable logic device (SPLD), a complex programmable logic device (CPLD), and a field programmable gate array (FPGA). When the processor is, for example, a CPU, the processor implements the functions by reading and executing the programs stored in the storage circuit. In contrast, when the processor is, for example, an ASIC, instead of storing the programs in the storage circuit, the functions are directly incorporated as the logic circuit of the processor. Furthermore, each of the processors according to the embodiment need not always be configured as a single circuit for each processor. It may also be possible to configure the processors as a single processor by combining a plurality of independent circuits, and implement the functions thereof. Furthermore, it may also be possible to integrate the plurality of components illustrated in each of the drawings into a single processor and implements the functions thereof.

Furthermore, in FIG. 1, it has been explained that the single memory 24 stores therein the program corresponding to each of the processing functions of the processing circuitry 25. However, the embodiments are not limited to this example. For example, it may also be possible to construct the configuration such that the plurality of memories 24 are arranged in a distributed manner and the processing circuitry 25 reads a corresponding program from each of the memories 24. Furthermore, it may be possible to directly incorporate the program in the circuit of the processor, instead of storing the program in the memory 24. In this case, the processor implements the functions by reading the program incorporated in the circuit and executing the program. The same applies to the memory 54 and the processing circuitry 55 illustrated in FIG. 15.

The components of the apparatuses according to the embodiments described above are conceptual functions, and need not always be physically configured as illustrated in the drawings. In other words, specific forms of distribution and integration of the apparatuses are not limited to those illustrated in the drawings, and all or part of the apparatuses may be functionally or physically distributed or integrated in an arbitrary units depending on various kinds of loads or use conditions. Furthermore, all or an arbitrary part of the processing functions performed by the apparatuses may be implemented by a CPU and by a program analyzed and executed by the CPU, or may be implemented as hardware by wired logic.

Furthermore, the medical information processing method explained in the above described embodiment can be implemented by executing a program that has been prepared in advance by a computer, such as a personal computer or a workstation. This program can be distributed through a network, such as the Internet. Furthermore, this program can be recorded on a computer-readable non-transitory recording medium, such as a hard disk, a flexible disk (FD), a compact-disk read-only memory (CD-ROM), a magneto optical disk (MO), and a digital versatile disk (DVD), and can be executed by being read by the computer from the recording medium.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

According to at least one of the embodiments explained above, it is possible to easily identify an attention grid that is used to perform a simulation.

### (Supplementary Note 1)

A medical information processing apparatus including:
an image data acquisition unit that acquires medical image data that includes a target organ,
a grid point cloud data acquisition unit that acquires grid point cloud data that is associated with the medical image data and that is related to the target organ,
a display control unit that displays the medical image data, and
an identification unit that identifies an attention grid included in the grid point cloud data on the basis of a display condition of the medical image data.

### (Supplementary Note 2)

The identification unit may identify the attention grid on the basis of the display condition related to a display range of the medical image data.

### (Supplementary Note 3)

The display condition related to the display range may include a display angle of the displayed medical image data and a position in a slice direction.

### (Supplementary Note 4)

The identification unit may identify the attention grid on the basis of the display condition related to the display range and a size of a treatment device.

### (Supplementary Note 5)

The display condition related to the display range may include a center position of the displayed medical image data.

### (Supplementary Note 6)

The display condition related to the display range may include an enlargement percentage of the displayed medical image data.

### (Supplementary Note 7)

The identification unit may identify the attention grid on the basis of the display condition related to a display color of the medical image data.

### (Supplementary Note 8)

The display control unit may display a plurality of images based on the medical image data, and
the identification unit may select an attention image from among the plurality of displayed image, and identify the attention grid on the basis of the display condition of the selected attention image.

### (Supplementary Note 9)

A processing unit that performs a physical simulation performed by using the identified attention grid as a calculation condition may further be provided.

### (Supplementary Note 10)

A medical information processing method including:
acquiring medical image data that includes a target organ,
acquiring grid point cloud data that is associated with the medical image data and that is related to the target organ,
displaying the medical image data, and
identifying an attention grid included in the grid point cloud data on the basis of a display condition of the medical image data.

### (Supplementary Note 11)

A computer-readable non-transitory recording medium having stored therein a program that causes a computer to execute a process including:
acquiring medical image data that includes a target organ,
acquiring grid point cloud data that is associated with the medical image data and that is related to the target organ,
displaying the medical image data, and
identifying an attention grid included in the grid point cloud data on the basis of a display condition of the medical image data.

### (Supplementary Note 12)

An information processing apparatus including:
an image data acquisition unit that acquires image data that includes a target object,
a grid point cloud data acquisition unit that acquires grid point cloud data that is associated with the image data and that is related to the target object,
a display control unit that displays the image data, and
an identification unit that identifies an attention grid included in the grid point cloud data on the basis of a display condition of the image data.

What is claimed is:

1. A medical information processing apparatus comprising processing circuitry configured to
acquire medical image data that includes a target organ;
acquire grid point cloud data that is associated with the medical image data and that is related to the target organ;
display the medical image data; and
identify an attention grid included in the grid point cloud data on the basis of a display condition of the medical image data.

2. The medical information processing apparatus according to claim 1, wherein the processing circuitry identifies the attention grid on the basis of the display condition related to a display range of the medical image data.

3. The medical information processing apparatus according to claim 2, wherein the display condition related to the display range includes a display angle of the displayed medical image data and a position in a slice direction.

4. The medical information processing apparatus according to claim 3, wherein the processing circuitry identifies the attention grid on the basis of the display condition related to the display range and a size of a treatment device.

5. The medical information processing apparatus according to claim 2, wherein the display condition related to the display range includes a center position of the displayed medical image data.

6. The medical information processing apparatus according to claim 4, wherein the display condition related to the display range includes an enlargement percentage of the displayed medical image data.

7. The medical information processing apparatus according to claim 1, wherein the processing circuitry identifies the attention grid on the basis of the display condition of a display color of the medical image data.

8. The medical information processing apparatus according to claim 1, wherein the processing circuitry
displays a plurality of images based on the medical image data,
selects an attention image from among the plurality of displayed images, and
identifies the attention grid on the basis of the display condition of the selected attention image.

9. The medical information processing apparatus according to claim 1, wherein the processing circuitry further performs a physical simulation by using the identified attention grid as calculation condition.

10. A medical information processing method comprising:
acquiring medical image data that includes a target organ;
acquiring grid point cloud data that is associated with the medical image data and that is related to the target organ;
displaying the medical image data; and
identifying an attention grid included in the grid point cloud data on the basis of a display condition of the medical image data.

11. A computer-readable non-transitory recording medium having stored therein a program that causes a computer to execute a process comprising:
acquiring medical image data that includes a target organ,
acquiring grid point cloud data that is associated with the medical image data and that is related to the target organ,
displaying the medical image data, and
identifying an attention grid included in the grid point cloud data on the basis of a display condition of the medical image data.

12. An information processing apparatus comprising processing circuitry configured to
acquire image data that includes a target object;
acquire grid point cloud data that is associated with the image data and that is related to the target object;
display the image data; and
identify an attention grid included in the grid point cloud data on the basis of a display condition of the image data.
